SEQUENCE LISTING (1 sequence)

SEQ ID NO: 1 - DNA, 38790 bp, unidentified
  source 1..38790: mol_type = genomic DNA
gtcctggtgtttctccaaggtctacttttatgctttcatttttctctaaccattttgtgttatctcccgaaccgtttaatttttgccagcacccaatcaatacttcttctactgattgttcgctagtttcaaatggttctaattttggattacaatcacatactgtgagataattgaaatctacaagtgttggtaaagcatgaatatataattctcctcttggttttacataggggaatcttgaaaattcaaaaagttttgttgtatcacatgttaatgatacaaaatcacatccttttagtttgaaagttacgttgccgtaccagtagttgaattttgattgttttgtgattacatttactatattttgttttgaatcactttcatcaccagtatattctgcagttatttctttatattctcctactttttctttgactgtttttgttgcagtggttgttgtttctgcttctccaggagttaatttaacttttttacatttaccatgaccccatttgtctcctccacagtatccgcagtaatctgcgtcacatcctccttttttcatgctgcaagttatttcgccttcgtacacttttttaggattgtcggttaatgttccagttttgccacagtttgggcagtagtttttccaggtttttgtgtatcttttatatgctgttcctgcacaacagccacatgatggcatcatgtttacggttattgtgttgggatctcctgttgtgacattttcagttacctgtttttctacttctttttcaacatatttcatgaattttattgacttatttttgtagtcaatagttatttcgctttcatcctgaattagattcggttgttggaaatatatcattgaagtattgggatcatcatctgctattccctgattagttatttcaattcctagtgtggatgctccttttatttttttaattttgagaatagggtatgattcgctgatatatttttcaacatctaaaatagtacatcctgggatttgacgtggtgtaatggttgggattttatttgaagaggttgtattttgtgtgtacatgaatggcatgtattcgtagttttcataatcttttatatagtgaatattttcttgtacaaatgttaaagaaacagtgtctattatttttagttcattagaattaaagaaaacatctttttttgttgttaaatattttgcaagaacatatatcaaatattggaagcttagtcttccacctgtaattgataagctgtttggtgcttttgtttgagttatgcagtacgatttgattattcttgctaattgtattatttcatcctgttttatgatgcagtcaatatggctgtgtgatggtaaatctgcacaatctacaattttaagtgaaatgttttcagagttattgtcttcagtgttgattattgcttctgcaagtagaagtatcatttgtcccatgcctattttaacattgtagactttgttttttgatgaatcttcatatatttcactatgtgatatttcaaacttgtttgggaagtatttttgaacatcaagaagtactaatagtttttcagcacaatcaataatgtcttgtatgtaaaaatcatattcaaataatgttggtggaatattattgtttacgtcaagattgtaaaattgtctaagtatttttgaataatctacttcatcagtattttctgaagaaattactggcattatacctaatgcatttttggtttcatcactactatatgataattcattagtattttctccaattacaacactttctacaattttattgtgtgttactccataatttgcaggttttagtaattgtatttcttttataatgttattattttttaaagaataatttgttttaaatactaatcctgattgtttttcaatagcttttaataagttgtattttgttattgagccttgaacattgattaatcttttactataatcaatactgtcaatatctgtttttgtaacattgaaaaatcctttcagtaatttgtttaggaataatttactgataatgattgtatttccatttacatgtgaagcataattgggatctcggatatagaatggtttacaattgtttaattctacaattatttcttctgcatctacatcaatagatttgtctatataatcaaaggtgcattcactattaataacaaacaaacaattatcaacaaagattttgtttccttgtttgaataattgcttgtctcttttaacatttgtaagtttatgtgatagttttaatgtttcatatccttgatattcatcagtaatatgtatttctgataattcagtatctaaaaattgaagaatattttcttgattatctaaaacaacaaccatactttttatcctcctttttcatagtaaattacatcagtcactttacaattgatactgttttcaaaattaaaatgatgatccaatatgaaaaaactagaatctacactaatacaattaggatctatcttgaaccattcactatatttttcatagaatacatttctattttcacaatcaattttaatctttgttgtttcaggtaagtttgttataaactctccttcaagtttcattagttgtccagattctgattcaattatttgtatgcttgttgattcatcttcacttaatttatacaaaataatgtctggttttacttttccaattgtggttattgttcctgcgaaactttttcttaatgggttaatatttctacttaaaccactagggattattaagttcacttcacaatcataacctccaaccattgcttctgcatcaattgaatcttctatatagtaatcaaaacattcttcaggtgcgaaaaagaaactaatactttttagaattggtgaatctaatgtgtctctttcaggatacaggtagtcacttacatatttcatgatttctgttgattcttcaaaatcatctccgtaaactctgaattttaatttgatttttttatcatcaacattgactcttatagggtatttcccatctgctccttcaattttatatttttctgtatcaaaattagctccacgaggtatttctgtttcaggactcatacttataaggaaatatttgcaactaacaccattaatatagaaatcgcagtcttctccactattttttgaataatatactgtgatttgcacatttttcatttcgacttggacttgagtgacattgtcaaatgcatcatcaatttgtaagaaaaaccttaaatcatttaatattgtgttgatttctggaaatggaatgccgaaattatggaattttccaccgaatttaaagttagtgtcatttctagtgatgtttatgctgtctaaatagtaatctaattcatcatcatctaatgttccaacaccacaaattagattaattgattcttcaacacttatgtctccagttatttctattccatgaatcagtacattattgttttctaatccttgccagttgaaaccttcaaagaaatgttttgtagataaagctaactctggaggttcaagtaataaatttgcagtgctgcctacatcacctaacaaatatctttttggagataaagccaatactggatattcataacctgcatatctttctttaagtattaaagataaattaccaaattctacagttccatagtcaaaatctatgaaattcccataaaaaattagatagattggtttacttgcatcatatgtaaactcacaggataactcttcccatatgtccttaacactgatttgattggtgtatgaatttgttgttccatttacaacacaaactcttaagtttttcaaacctttttcaattttagaagcatttaacagtcttcctaatgctgagaaaacataagtttcaccatgttcaaggtttgataatgtgaattctgataaattcaatattgaataataggatttatatgaatcaggaatgtttactttcactttatctgtgaaaacacctaataataatggactggtcatttcaatattatgctctccactacttttagagtataaaggaatatttgtatcaaaaacaatgtttccactatcatctttttgccattgtgattttgaaatttgtaaaatattatttgataatcctggaacaggttcttttacagttaatcctccaaaattcagatttagtgttaaatctaatggagcatcagtttttaaagttatgtttaagtatgttaaatctgtaactgtatctgtactaatttcaatttctttgttatttgcatagaaatcccatacttccaattggggtttttctactacaaatgttgtttttgttgaatggccactattgttttcatcaagtgtgattgtaaacaatccttcagatgttggagttacatagaatattaattctgctttaccatttgttgttgcttgtctccatatgcctgtggtccagtcataatatggtgaatcatatgtgatttcacagtctccatttgtgattatctttatgtttgccgatatttcttctgaaggttttgttccactggtttcaaccattgtgcattttacagctactttttcaccgtaactaacaatatttggttcaatgctaatgcttggaatataatttccagttgtatacactacaattactcttaagtatttcatttcaatgtaacatgggtttgtagatgtattttgtggtaaatcaaatttaacatcaaaatttggagtgttaatgtcttttccttttagattcaaattattaaattgaacttcatattctccaaaattatttctaggtggggcattacctgtttttgacaggtttaaactacttaatatgatttctggatttgaaaaacttccatgtgcagtttgacttatctgtgcagagtatgctaattttctatatgaatattgtactattatactcgttactttagctgaatctggaattaatccatcatatttaaaatcatatccacgtatggttgatggatgttttcttgaaccatttcttccagcaatcgcatttttatattttgctccaggatctgcataaccccaatgtcccacagtatttaaatcatttgtaatattttcaatatgatcacatgaacgataaggtatgttgtttccttctatattttgaactttactaggatatcttaaaatactaggcaatttaataatccctccatcattttttttaatttttaataaaaccatcatactctgaattaaacaataaacagtttaattgaaagctaatgtaatctcctttacttaccccatcaggaaaagcaaattcaacactcacatatttagcaccagcaggtatttttgatgttttaatagcacatcttcctttattacccataccttcattagcaatatccataatttcctgattttcaattaatttaccatctttatcgaaaaatacaaacccaacaagtatgctttcaatagtgttttttgcatcataattagatgaaaacacaatactggaagcatcatcaggtatgtgtactccttgattgttctgatggattaaaagaaaaaaaggagaagttaaatcaacattctccgagagatatgtttctttaactttcaaagtataaccatttctaaaaggaacaacttcaaaaaaatcactatatgggttgaaattaaaatcaaaatgttcacaaacaaacaaactagataaactagattccatacaatattgaatatcttcactaaatctgttagttaatgaaacatctttacttaatttaaatgaatatacattgtaagattcccattcatcaggttttacattatttaatgaaaccatacatttatgtgattcaaaatcgtttttatcttccagatatccatgtttgttacttatgatattaccgaagatttcacattcttcaggattaatttttatagtttccattatcttttaacctctcattcttttaatacgattgtctcttttaattaaagcttcagcaatgcggtcaattaatttggaatcagttatcacttcttttaaagttgtgattattgatttttcatcaatatcttctccttcaacagtaacattaagattatgatctattgttaaatgcacatcatcagatgtataagacactaaattatcagcagtagtattattcctattatgttcaatattctggaagtatgcattttgatttgttttcattaaatcaataatattatatattccatctgtaatgattccgaatacatctttagaacttcctccaggacttgctgaaccaaaactaactccagaacctccgcggaatattcctccacgtttttggaattgtgtcatatcatatatttgaccattaaccatagctgctacatggcctattccattccatgatccatgaatcatgtgtcctgataatcccaacatagatgctatttcaattatcatttctgcaccatcaaaacagttgcatcttacttgatcccaaacttgctgattactatatcttgaattataataaaattcataagttccagggttttggaatcctcttgcagttaagatttttcttagaagattttcaaagtttccaggatttaattgcggttgttttccatcacggaaatcttccacatgataatctgaaggtatttgaattccaaggaaccatgggtctgctatcctccatttgtatgcagtattcattattttattcacattagtatcaggaattcctgcaaaacaagtatttggattaacacaaccattaactcttgccaaatcattaattacgcttgttggtaaattataatctaaaagatttaaccatgatttgcttttattgttttgaggatgttttactttagaattagtgtcaattactctgccttttccttgtcctaatcctccagcagcataacttgctattgtgccttttgtggattttccaaagttaatgcttccaccaggtaatgttctgcctccaattaatcctcttgttgaattagttgctgggcctgcaggcagtccactactgaaatgtgcattggctaattgattatagaaacttgcaatacttctatgaaggctgctgaatttattgtatgattgtgtttgtatgcttcctgcagctgaaacaatattgtctttcattactccccatgcattagtcattttatttgttacatctattgtggagttacgtacttgatttaagttctgggttgttgttgatttaatattgttccatgcttgagtatttgaattactcatgcttgttaatgcagtggttactccattacgtgttgtttggaagcttgttactactcctgcaactctcatttgaatagctcctgcatttaatgctattcctgcagttaatgttgaaaagcttgttccgatcatattgttgcttgcagtgatgctttcactagctaatgatacttcttcaacaagaccattatattggtctccagtttcttctgtacctgaattattattgcttaaatcaacatttgcactatctgtttcaacttcagtgtttgttgttactggagttgtgatagttgttgaaatattatttaatgcattttcaacatcagcagtattgaatccatttacaatgttttttccaacatttttacctgcttcatatgctgattttcctttgtctttaactctttgtagcatgttaaagaattctagtactactttttcttggattattcctggtgaatgtattcccattgcggaaagcattccgtctacaatttttttaccgatttgctttgcattttcaactaattgtgaacctgcagatagtattcttgagcctatgtttaggaattcctggtatactttttgaggtaattgctttatccaggacattactccagatactacttgtgatgcttttgttttagcattacttacccattgtgctcctgcagttattactcttacagcaactgctaaaagatacatttttattttatctggcaattgtcggataaattgaactactcctgtaaggaagtttctaccagtattaactgcattattccacattgactgagcccaactaacaacacgattaatcacatttgataatattgtgctgattgttgattgaatagctgcccaagcccaagcaattgcttgttgaagacttatttgaccagtaattaactggctaaaaattgttgccagttgcatgaataaattgaataatggagttacaatatcaagtacaagttgccatgctggaccaaatacacttaataagaatgatgcaattccattgaaaacatcccatattatggtgagtatgtctattgcaagttgaactctaactttgaaactttcaaatactgctcctattgcatcgataactgctcttgttccatcaactttaccttttgcactttctgggaatatttcatcccatattcctttcaaccaatctacaactggtttgagagattcattcaaatcagaccatgcatcttgaattgctttgatagttgcttttacatcaggatgatttatgaatgcatcccataatctaccaatattattctttatggcttcgagcatggttcctacatctttccaccaaccaaaagctttacctacttcatatattgctactgctattgcaactaatgccacaacaacccatgttaatggatttgataaaattgctcctgcaactgctaaaaaactagccgccaaacccatatttgctatttcaacacctaatgctccagcaatataagttacaaaactagtcgcactagcagctattgcgggaattactgaaccaagaattacaatacccaacccaactaaagctgttgaaagtacaccaatagttactgctgctttagcccaatcgggcatgcttgaccatgctgattgtaatgcttcaaccatccaagtaatcatttcaacaactgttataattatgggagttattgggacaaggatagattgtattaatttgcttccagttactgttaatgctgcccaagcatcatctaatgttacaatttcttgtgcggactgttcaaaacccatatcgctaagtgttttgttcattgcttggagtaatccagttttattatttatatctccatcccatccatttttcatgagcatgtcttgagatatgcctatttcctgcagtcttctgaattggccatccattgcatctgatactgcaagtattgcatcttcttgtgttcttccttcttttacgaaagctgaactcattactgcagttgttttagtaagttcatccattgaagctttaggaagttttaatttcactcccatttctaatgcggctgcacctactgcattcatatctacttttctgaagctatcttgcattttgtcaacagccccatggaattgttgtagttctccttcagtcattcctaatctttgaccgaatctttcaaaacttgcagctgcatttattgattctcttgctccttgaaccatactgtttacaaggtcaaaaccaatcattcctacagtcatacttgctgctgttctaagaaatccaagacctccacctgcagttttagctccgttttcaagtcctccgaaaccagttcctgttttttgtgaacttgttcttaaaccttctaaagctaattgtgcttctcttgcatctcttgtaacattatctaaaccagtagtatggaagtttagcaatgaatttacttgttgaagtataccaattaaaactaataatattgcttttagtagttctgctgatgctgttgttctactgaatcccgaaccatcaattgaatctaaggctgcatctgttgaatgtgcacttgttctagctcgatctaatgccatgtctaattgtgctgcacttgatgaagttgctgttaatgtgtatccagttatactatttattccactgcttaaactaagtgcactggatgaagcttgttttaaacttgaagataataatctggtactattatttgaactattcatcgaggaagataatcttgtagcactagatgatgctgtgagtaaattacctgaattaattgtgttgattgaagtgttaagtctatatgcactattcatcgatgtatccattgaagtggttaatagttttgcagaattagataattgattaattgtattgggattgattgaatttaatgctgtttttgtagaattagtgcgatttgtaacttcagttaattctttatctaactgattagcagagtttttggctttatccatacttgaggaatcaaattgtcgcattgcattttgagcttgttgtgctgctttttgaacttgctccatttgtgcttgtatctcttttaattgagctttaacacggttttccagttcaattattaactttacaatattattgctcattattctattcctctgctaatttcttgtttttttcttctgacacggtcttttagagaatgtgaagcatgatttgtacctgaagattgtttatcctgtttatcacgaactaagggtactgcatgattcaagaataatttctgcctaatagttaaatctgcttggttttctgctaacatataaccgatgtcttgaagagttactacttcaatagctatgttttcattcttcttcacgaaattctgctatattattatccattgatttaaggttatctaaaccactgaaaattaatgcttcatttacaattaagtctaatgttcctgcttttaattgttcaaggtcttttttggtgaatttatcaggattgtctgcattgtctaatacttttaatgcaaggttcatttttgcatcatatttctttttttccattgctgcgagactggttttgaattttgtggtgtcttctgcaaggctggtttgttttgctttgatggtggaaatcatgtcgaagtttcctacatcacggtatgctgatgtgaattggttgtattcaaggtcgcttatttctcttattgctacttcattatcgtacattggtagttcgattatttttgtgtttttgattccgccgatgatcatttcttttgtaagcataaatatcacatttttatataataatttttttttgaattttaaataaagtaattgaatgagaataatcatattctcatttcatttaatctgtgtaaacagtgattgtttttgttttacttgcaccatcaattaaaatatgtgctggttctactttagttgcagttggttcaccagtcaactctatatcatacactccatcttcagcagtaagtgttacttcaccattacttgaagattcagcatcaacagttgtggatcctttcattaatgatactgtgtaatcagttgttaatttagcaccttccttatcaacaagttttattgttgcatcataagttgggttgggatcactgtctgcttttcttgcaaataatgctccagctaaatttttataatggtccattgttgattcaactgtttttgataaacaaacaatacttaattcatatttaactgggtctgcattcattgttgcagatggttggttgataatacacattggacaatataattcgaattttttatcaccatttctaatgtagaacattaattcaaagtattctcctgcaaggtatcttcttggtccttcctcacttccccagtacatttccaagtatttttcatcagtatcatctactgttaagtttaaaccaatttctcttttagctgctcttggtattttacacataaaccttgagcccattcctcttgcatcatcagtattaacattgtttttgatttcaagacttattttattggttttacatctcatttcagtccattcatcttcaccaaatcttctcattttaacactgtctatatggtaaaatgataatggtaattttccaaagttaaactctgataatttttttaatggttgtttactattgatttttgatttgatttctgctgatgcagttaagaattctgattctacttcaatacttaaactgtccatcaccatacctaaaatttccatctcgtaggttgcacatcctgccatcacagtataagaaggtaagatactactatttgtaccgtagataatatctccttctcttcttcccaatactgcttcaagaaggtattctaatgtttccatttgtaagttgcaactgaaactgttttcaggtacatagtaagcagctacaccagattgataatccctacttactactgaatcatattttgtaaattgctctggtggagatacatccatctctgatatttcaatatcaatagattctccactaaggtctttttttgcaaattctccttcttttactaatcttaaatattttagttcttctgcggctgttaccatattctttagcctccatcacattttgaattcttaattttaaatcttatttggaatgttacacctgcactatacacatttttctttttacccagaccatatggaacccatccgaaatccttggttttaatattgaaaaattctaagtctccaagtaatcttgaacttattattctacgttctgcctgtgaaactatacttgtagcttcatgaatacctatcttaggatttttattgttaagcacctgtgaggataatattatttctccttcaaaggtttcacttaaaccagtatcagtaatatttttaaatggttcatctacccataattccagataaggtaattctggtgttcccagacttacacccataagagataaatgtttaatagtgccatcttcaatcatttcagataagaccagttggaaaccttccattagtttatcataagcttctatgaaatctttcataatcccacctcttttaatgtttcagacacatatttgttaatttttgattcagtagttttaactgcatgaggataataatggtatcccctgaatgctgctacttttttaggtttagggacaaaaacatctttaccagatttatccacccaatgcagggcttttttatttttagctgagaaacttccacgaccatcatgaacatatttttcataacctgaaggttttcctcttgcttttataacatacatttcatctgtttcccttcttgcagttattgcagttaaaagattgtaacgattgtattttatcctactttgaaggtaagttttagcatctttacttgctccataacttacctttttacctaatcttggaggtacccgtaatattttttcttgcagtctttcccatcccgcataatcaaaatcaaaatgtatcataaaaaatcatcaactcttataaaaaattaaaaaaaatgtttagattgagaaaacatgaatcttagattttttaatgtaaggcttcattcttttttcaatgtcttctgtgaaaatattgtttactgcctcatcaaaatcaaaattttcatgatcagtaattcccaagtcctgtcttacagcatggcttctaataatgttactggttaattcaataatgatttgtattacatcatcaggaattttatctggtatatcatatttctttttaacccatgattccattgcagtgtaataatcattaatataattatctaattcagtagtttctaaattaaataattcttcagtatttttattagctccactgtactgtttgattttttttaaggtgttttcatccaacataaattattcctatgttttagtcattgtaatggttaatgtagttgaatcatcacctacagtataattacctgtgctttcaaagttttcataaccagtacatgttgcaccatatgaataaactccataaggaacattacttattgtacagcctccagcactaccagtaccgttacaagtgtaagttttaccatcagtaatatttgttaaagttacaatagctccttcaacaggatttgttccatcatcaacacttacaccaatatttttagtactagttgttgtgctggagtttatgcttcctttatgtatgcaagagcagatgcatcagaccatgcaaactgaatatcagcatacattgtagaagcaatataatacttgttagattttaaatcaaattcagattcaacaataatattatcaggatccgccaaccattgaatgttttctttatgagttaaaattactggttttttagtgaaaccattacgaagagtactgaaagctggaattggtactaatggtacttcctcaataatgatgtccccatctttagtaataactacatcattaacagcatacttgtcatgattatctgctacatatctccatactgctcttttaaaactgtatggtacaaatgcagctaccccaccatcatttttatatttatcagggaataaatccaacattctacggaattcttttaacggattggaatctgttgcagttaaatctattgtttcttgatctatatcagaatcatcttctaattttttcaagataccatcatttactttatatgaagttgcaacattagattcagtacttgcagtattcccatagattaatgttctttctaatgctctaccatttgcactaccaaattgaccagttaaagtattcatgaaattcttttgctcaatactgtcatataatacagttctgtgaacaccagttaaagcacgatattcttctgcatcaaaagccctattagtgaatgttgggtcctgttcattgcttaaagtttgtggtgtaccactgattcttccagcttccaattcaatatcaaaactcatcatgtccaattctcttttatgattgtgagttggaacaactttagtcttgtttaaaaatgctgtttcttcttgaacagcttgcatgtatttatctgctttttccgcttgaagaactcctttaccgagtttcccacttccttctccaatatctacaaatttaaggataaaatcttcattatgcatgattttatcagctaacgcttgttctacttgtttaattgtcattttatatcaaaccttctcataaaaaatttaattattaagtataatatcttctttttttctaccaggtcattccatttgaattcctacccattctctcaaggaatgatttttcagaagcattgccttttgcaaggtcaggatcaatacttttactaacagtagcatctggattaacagcaccatcagcagtaatagtagtatccactttaacttctcctccatcatcatcttttggaggttctgattttttaacagctccaggaacaggatcctcttttggaggttctggtggtttttccccaccttcaagtttagttatccttgcatccatagtatctaatcttttatcgattttacctagtgcttctaaaattttattttcatcttccacaggttcattattttgtgctggaggttcagttggttcttgaggttcagatttagcaactaaaccattaattaatttttcaagaataccttctgaaacctgcacattattttttccattgtcttcttttgtcataacttccacctcttctggtgtgatatattttttcacaaattcttcatcatcttcatatacttcaaaaaccgccataggatgtgatggttgatcaacaatactgattgttgatggtttccaatctttaatgtctttaaatttcaaagccatatttaacctcctaatcctctactcatttgagcaacacttttatatggtgcagccagtatgctgaaaccattgtattccccatctcggattgcttgttgtatttcctcatcagttacatcaacagaaataaaccaggatcctttggggtaagtgtttcccctgaaagtggttggtgattcaagaatatatgattctaaaattcttcctacaggttggagtgtgtgctgtacatcaactcctaatcccaggcgattgtatattaatgatgcttgtcttattgtttcttcatccagtatgtctcctgttgcatcagggattcctggtatgcagactgcacctttcacaatcatagttttttttgactccccatttttttatattggaaaaaaattagttttgttggagttttttttagtaaaaaagaaaggaggattttaaaaaaagataaatgatgttaagttactaaaataaaaatatttttttatcccccttcaatatataggagtggaaaaatagaaaaaaacgaaaagttaaataaatttattcaacaattttgaccataggtttaaattgaggatatttatcaagaaaatctaaagcatattgtatatgatattccctagcttctttgtgtttcccttcatattttaatctatgagttacagcccgttcattctgatccaaagattcaaactccggatgttcaatacactgaattaatcttaatcgattaatattaggaataacttcacgtacaatactcattaatctatgaggtaaacctgaaggtgttgtaatgaaatctcttaattgatcctcagaccaatttttaacaaattcataattaggaatcaatttttcaaccatgataatgaaccccagtatttgttagtatattatatataaaacttcctatataaaaattacgatatcatttcccaatgagcaataaactctgatttttcacgtattaatcttaaatcaacatttttatcaaacaaaatttcaaactcctgaggagtttcagttattccttcaatatacagaattttaattccttttggaatgacaatataattaatatactccccatattcttcaggtttaacattttcggaaatagaagttgataaaaatgaatctgaatgataattatctccttctacaaatttagtcattggacttttttgtcttctaactacaacaagactttcttctaatattttacctttcttaaatactgattttgaagatttaattgctttattaaaagagatataatattttgaattagctaaatcctgctcccataatttagaatattttcttttaatacgccacctcttaactgggttgagctccccttttatttctctgaaataattattcaaactataatatccttcatcagaatatgcttttatgaatttatactcttcagaagtcaaatcttcttttttaattccatatttatattcaaatgccaatttatctttagatgattcaccaacaccataaactgttttattaccaattttcactgctttatttttagctttactaaatttagtgatagattcatttaatttaaatttactgaaatctctttttgaattttgttttggttttgaagaaatattaatcattactgtttttcttttaacattttcagtgattttacttcctgtatttttaatcttgttacctatattcgatatggttctaattatcttattcttaatcttagaagttatcttcaaagaattttgattactgctacttttattcttaccatgtaagtaagaattagaaggaatattaaaagaacttttacttttaagattacttggagtactattagtatagcttaaccaacatctgcaattagcaacattctcagcacctccagctaaatccccaggatacatcatctcagtacgataactaccagtaataataaaataatcatcaataggaacactctcaataaagttagcacgatgccaaattctatgttgtctagcacgaccattcatccaaaccttatacttataccccttatccaaagcatgtaaaaaactcatcctagactgattagtatgcaaagcatcacgaacaatattcttagcattcttacgatacaactccccatacttctttttaatctcactatcagacaacttactttcagcttcaatcttagcttttaacttataagcatcactatcaaaacgtgaagataaaatcttagaataatccccaacagtttgatcaacactaccttgaataatcttagaacgctttttcattaaagtattagcttgaccttgcaaagcagtatcaattatgttctctgcaatctttttactattcccaattttatttccatcaataatattatttaataatttatctttataagaatcaatacttttatcagctttattcgtatgtttaaacaaatgattaaaacttatatctcttttaaccacataaagctgatgcaatctttgagttaaaagcagatactgaagtttctttttagaaatcataataaaccattcttattaaaatcagaattcaaagcatcaatcatactatccaatttaccttcagcatccattggagcatcaccatacaaaaccttatccaaagactgattattcataaacctacaattataatactcatcatcttcactcattttcaaatcaaaagattcaccaaaacgattaacaaactcccctaaagtcactgcaccattttgcagtaatttaattccacgatccaatactttatcttcctcatcaaaattaacaggcaaatactcaagtttatgtgtaaatattccaaattccttttgaataatagtcttattaatcaaattagccatcctcttttgcaatgtagctactgtagatttactataatttttaagcaaagtttcagtacgattactagcaatacctgtagactcagaatcacctaaacgttcacgaggtacacgatgaatacgacgaatacgatcagccacactacctgacagttctaaaaaactgccttcttttttctcatcagcaattttagtaacattaacactaacattattttcttcactgggaatagtgaaaactaaagcagttccaggctcattggacacttcacgaaactcatgttccaaatcctcttcaaaatcatcaaaagtataatcttctttttcctcaacattacctgtaacagtaataatataattcggaataccatgtgctttaaaatgcccttgctgatactcaataatagcattatcagttaaaatagcatccaactcggacaaatatttcggtctaccataaacaagactttcattacttttaagattaaaccagattaaatcatttgcacgattttcatctgtaatattatcatcccacaaaccagtttcacgattcaattgcttaatattgtcaggatcatataatttgaaataattttctttatgccctatcttttgtacaacacgttttttatcacgacacattctcaggtataaactgcttacatgattaacacttttcaactcccctttttccctaagtagttctaatcctgcaaaaccaaaagcttctaaatcttctaggaatgattctatttcctcatcaaaattaaaatcatttaaaaaatcatctaatccttcaggtatttcttctccttctttagagattatttttttacctgtgaaaattgcatcttcacatttaacagtaatacaaatatcatgcaaaccactaatatcacgcaacttatctaattggaacgggtcatacgctgggtcaattacttctgtaccatatgttaattcatctttactcatttcttgagatttaatctcatattcatttaaaacacttttaatcactgaatctcttaaaaagttacttttaactattttaaccattattaatttctccttctacgatttctttttcttaaaactgtaggttttgtttgaggatataaaccttcatgtaacaatgatacactatctacacggtcatcatgtgttgtttcatcatcagctattattttctctgatgggaatttcacagcttgtttcatgaaatctttcaaccatttaccacgaacaaataatatccgtccattattcatcccccgtatagttctacttgctcttattaattttgatttaggaacacgaataaaattaggatgatagtcttcgaattcgttttcccagtatcttttaactattttacctgcagctgcaggctgatactcaatccaattatcatactctgaatgttcatccataatccttgtcatgtattgttctaacttacctggctttttttgtgtgcttttctgattatgcactacacctacttttccttgtaatactgttgaaaaactacatgctgtataatcagaagtacttttttctgttgctgcaatatcccatgtaattacttcttgaagaatatccttttcagttaataattcattaaactcttgtttgctaattgttgctgcttcaattgtatcataatcaaatacatctccagctcttatttcataatcccaattaccaatctgatactgataatctgcttttgataattcacgtaaattagctaagtatttttctttgtcaatatatggattatgccagaaattcatttcaaaaaaagggtattgtcctttaacaaatttttcatttaagtatgttgatccatctgcatcagcaggattactaatatagtaaattgctaatgggaaagtcattaacttatcagttcctctgagactacgattcaggaattgcaggtttactttttcaaattctgaagcttcatcaacaatgattttatgataagcacgacttttgaatttctctttgtctttttccagaagcatatatgaataataaatccgtgcatcattttcatggttaatgaaacatcttttactttgattgtgttctatgtaatcgaatggttctgtccaatggtccaggtaatctactattcctccagtcgctattacattatcataagtagaccgtagaattaagcagcggtagtatggtacttcataatgttgtaaggctaatactgctccaagcatacttttaccagaatatgcggaacctccaattagttttcttgtgtggcggtctgcaattgcatataattgtcggtcgtatggtgttactggaatgtatgggttttcaagtattgttcttctaatcaactttttttgttgttgatctaagtgtattttcttgtaatctactctcataattcatcagtgaatttttctaaatcatcgttaatggttagtaattcttctaattctttgtcgttcatatggattacattatcattttctttggtggatgtttcaatacttccttgtatttgagtattattatctttaatatctgttggttgaccatgagctagtctgaaatttctatagattatttccgcagatttatttagatttagaaaactatttgctcgtgtagttggagcattctcattactattttcttttagttctttttgtatttcttttagtaattcaaaatcatcattgaagaattcttgaaacttagcatttccttgtttgaataattcatagttccactctctttctttttgatccatatgattatcatataatctgcatctttccacccaattccatcttgaagagagtgtttttagttgacttagtgaaggtatgtttttttcttcagtatgcttcgatttcttcaattttattatctgtgggaatgttcttttggaacctaaatctctgtattctttgaatagtgaatagcttttacttggttctcctttttgtctttcccatgcttcagtcataatccccccatccttatattttttttgaacattattgtaatgtttattcttttttgttgaacacgaaattttaaaatacgggattttttattttcatagtgaacaatataatattgtttagttttgaattttgaacaaaaaaaagttttattagttttttataatagttttaggaaattcacaataacaaactgaaatccatcaatcaaaaatacaatcaaagcaccaacaattgctatgaatactccactttttattgtgaataatcgattatcatcattagcttgttcttcctgaatagccaactgttttgtttgaatttcaatacttttatccattttctttatcagtttttctaattgcttattttgaaactgatcacttgcttccagtttacttatacgagattcctgtttacaatatttctcatgtaaatcttgaacttgattatgggtcattattgtttcctccattgttagtggaggtgtaagttcttgaaatccatccaactatcccccctaatgctactgctgctagttcgttgtttccaatataggtgcttagaattcctataattattattccaattattgctaatgttgtgttgttgaagttagtcattattattttcactcctaaaaaaaatttgtttttaaaaaaaacattggggaagggatttgaacccctgcgatactagtgtatcattagattagcagtctaacgccctaccaggctaggctaccccaacattgttggtggagggaattgcaccctcaacttttgagtggttactaagttacaataaaaaataagctattaccaacatcatgaacatgtattaaaggagataaatatttatattatatttatgggaagaatatttttttttaagatatgtgatattaattatttaatctttggaggatacctatatattttaatgaagaaaaaaatccaatcattctattattcattaaaaatttgctaccattcccataataatttttaagaataataataaatgaaaagcgaataataaacggataagaatattatttttaaattgtcaaataatgataatgcgaagctaattgtacaataacatatgataaaatattttttataagtgttttattacaagaccacattttttacaaacatactcatcatgccactcatcatagtaaacctttttgcaatcttttttcagtttttcacaaacaggacactctatttctgcatgtttcattttttaagggaaatacacacccgcagtttgtgcaattatattctgcacgagattcatccaaaacagttaaaggggattcaggaaacttaatataacaagatgtcttatgatttctataatgtttttcttgttttttcctttgtttccacttccagttacaaacaggacactctatttctgcatgtttcatgtttttaaaaaatgtattagcatccaaataatattatcctcccatatataattataataatgtcttttatttttttttcataaaaaaagataaaattataaaatattttatccccttcaatatataggagtggaaaaatagaaaaaaagattaaaaataaaaaaaatttaatcacttaataaataactatcaagaaaatgctcaccacgactgatatgtcccttacttacaatccatttaatcatgggatccatcaaacctttttcatattctttaataaaataattcaatgtccaaaaagtaaccttttcattaaaaattatcctttcaaaaagttctttagatgtctttttaaatttttttctatgaaaaataatactgtcaatttcatcaatagttaaatacaatgcaatatcttcaccagaaatataaatacattccttatcaacatcataccaataatacttctcattaggatttcttaataatttaagatacttgataaaattcatatttttactcctattctttttaattctcttttcacagttctttcctccaaaccaaaatcactcaaagcatgctgtgaaagaaatgttgtaccaggctcccttaactcattactaattaactccccatcattaattaatttcctacgtttttgttgatactttgctttctgctcacgtaccgcccaggtcctgcaagcttccctgcaataacctgttttattctcaaacttaataaatactctgccacagtatttgcatttgctgatgtagaatcttgtttgtggatcatttaatgttttcttcaataacatcctatcatctttctaaatttttagatagtaagtttttcttcacttcaaaaacaaacatgtgaaaaggtaaaagtttaccctgatatttcacatattcattaattacttgtgatttttttaatgcttcagttatagtttcttgttttaattcttcatttatgcattcaaaattggagactgttctacttaaactaatgtatactctttcatatagattaaggttattgtttttcacattctccaccacaacaagaaactgaattttcagaagtgttgaatttaccatcttcaatcatttcaatcaactcattaagaatacgagtaacatgcatcaaagtatgaagattaatactatgaattataacatcatcatactccatgtctctcaaactacctgtgcattctttatgaatagtaaaagattgttcttcaaattccctgcttcttgttttaataaaatatagtaataatttctcatcaatcatacatatttatcctccatgtattttttaacttctttcataaaaactcctgattacaatataacctatcttaatcatcaaaaaaatcagtaagacataacactaaacaacaactaataattacacttacactaaaaactacccaaccatccatcatactatattctccccccattatctccaacttgttattaactcatactcctcatgagttaacttctgttttaacctttcatcaaaacttcttacatctccaggattattctcatcccaacaagactcaatcttggcagttaattcattaatttcttccttattcattttcaatatcctcctatttaattttgaaaggttgtaatggtagtgttggtctttcaaatataatctgaaacaaaacagtatccccacatatatctggataaatactttttaactcatgtttcaacaaatcaacatgcctataaccttcaaaccaagcatgattatgtttcaaatctttaaatttaacctgttcaactctccgtactacccgaagcagacaaaaacccgtcccctcaaaccaacattctacaaggtcaccttttttcaatcctttatcagattttcttattgtagacatttttttcctacttcttaaaggttcaaagaaatacggtttaaacttcaatgttttcatttttatcaacttccttttttacatatctttgcttcttttttatcatcatccaacactatattatgttcttttaggtatgattttccaaataattcttcagcatgtttacataacagataagaatcaattactccttcagcagatattattccttctttttctaatttttcaagtatatcattacaactcatttttggatctcctttttattccggactggttgtctttgacttggttcaggaacaccacacctcatacgtaccttttcagcaaacttaacaaactcatctgcaaataaacttattctaaaccacatcaactgccaccttaatataatatgttcctcatcttctttcttcatgattccaccttttttaacctagtttggaagttatttcccccataacgcctaacatgatcttcacaaatgattcttttcacaaaggaatctttttgatgtcgagttaatccataccttttctgcaactcatcaacagagatatttaattcatgtttataatctctcctgaatttcaaatatgtttcttcacctttagaaacatcataatccgtttcaacaatattaaaaactggtttattcattttttatctctccttaaaaaaatgtaaacataaatgtttacaatttaaaaaatatttatagtgaattacgtacgtatacgtacgtacgtatgtgcacgtacgtggtttcctacaggttcttcggaaaaatgtaaacataaatgtttacaaatcattttatcaccaaaccataattccgaacatcataacccaaatcatacacatgatccaataatgacttatacggaacttcttgctgcttactaatattcttcagctgatctttaccaataaaaccagcattatcaacaatcctattaactgtacccatcaccccatcaaagacatttacacttctttcatgttctaaacgctcagcacgaatcttacataatcttgattcacgagcattaatctcattttgcaactcagctatctcttttctcaaactattttcctcatcttcagataaatcaatagctaatcttaactgctcttctaaaaactcacttctggttgtccccagtttgtttttagcttccctccatacttcagaatcaaccatgaatgaaacctgtattttttcactcatacatcatcacctaactctttcatgacagtttctaattgcatttcctttgcaattaaatctaattttaaagacctgatttcctcttttaactggtccttttcaattaacctttgattttgtttactcacatacatatcaatgcaatgttccacgcagaaacgtacagttaaaccatacttttccattaacttttttgcccttgtagtaatcctaccagataccacttcagggaaattatcctgagtattcatactcttacacctaacattttttctgcttttttaatatgtttgcatttataatgttctactttttgatgtcggtagaaataatcttcacaactacaccaccatccatctatactgtcccagttcacagtattggatccagtatttcctgatgccttgaattgtatgaatattatttctaccttaggttccataattaaacagcaacctctttttcttgatttttaaaccagttatatgcttctttgttttcagctttggtcagttcaccatctttgaataacttcattctatgagtattaatcatagatggagttatttccagtgaactgtcctttttatgaatatactcgcggattttctgcacagcaccagtagcctcaactttctcagttcttgcagaactacccttagcagttttaacattaattttatcagaatccacaacatctttttcaacaattagaaacatgtttgctaataaatatctttttaagtaagtaatgtaactgccttcactttgcattacattcatacctttatttaaagcaactatttcaggcataggaacactggtgatgacagattccccaggttcattccaatttctgattttcaattgagccaattcatttgtaaatgaaaactcaatgaataactcctgttcataacattcacaaaagattgcaggcaaaatatcttctaattcatagtacttaaatttcccaaatttattgtacccacttttaggtaattttttatgcaggagattcttttgaattcctgcaattttctgatatatagtcataaaaaaataatccccctttttacatattcatcgactttttcatcaaatccctaatttcttttaactgttcattttcacttatttttctaaacatggtgttcagaatatattcatcgtaatctatatttttttcagcacataactcttccatcagtgctttaactattctaacttgatttttctgtacaatatgtttaaagatcatatcaaaaaatgaattcaattctgcaggatttttagcatctacattatggtctaaaaagtggattgcattagttaatttctcctttaactcttttttgtttatgatttcttcagtaaatagttctttctccattaaaattcctcctcaataatctccaatactttgttttcatcaatattttccaaagatgcttgagagtgtggaatgaaactttttgacacccagtagtcatgatactggatttcaccaccacacagttcctgtaaagtattcataatatcacgtgtgttctcataagtactgtcatgaaaaacaattacagataagaaaataccatgtaagtttccatgagcagaaatacgtccgcccattttttcaacaattcctaataattgattgtaaaacctgttttgctgatcataagtaatcatactaaacacccattattataaggatccataggccctactacaaggaatgcaaaaatgaataataaaattattaaaattattccttttgcaaagattatttcaacttcatacttttctctccaggacacttttttatgaagtctaactggagtaggtttattgttgaagagactcataattgagcctccaacactttatacttaacttcttttttcataccaggagtattaaggaaatctttcaattcccttaatctggtttgttcttcaacatactctgcatactccccgtataatgtttcgaacaattctctttgttcaggatattcctgtgctagaaatacgaacagttcatctatttcacagtaatctttgtactgtgactggtttatacgatataacgcattaccaatcttgttttcatttttccaaaattcaggagtgaacattcatttacccccgtaagctagttcacctgcacgagcaaactccaacatttgatcatcagacaattcatagatttgaaattgcttatattcatcagccaacctatgatctatttcttcctgtgtttcataatcctcagacaataatatctgttcaagataatcaaactcgacctcatcttcaggtggagtaatcgtatacattgagttgttgtatctgcaactcatacgcccacactcctgaatttttggagttcttcttcatagactctgttttgagcctgaagcctttttgcttttaaaaatacatgttcaatttcttctttggaagttaaccttccaaaaacttcaatcttattattatccatattaccaactcgtggattttttagaagatcttcttcagagttgccgctctgattttttcttcttcaaataactattatacttaatactatataaaggttattatatttacctttattaaaggtaaatatatatactgcaaaacagaatatattatataagaaatataaaaatggaggtatgcctattttagaatatacaacaaaattaagtaaaggcggacctaattctattaggtcaatagtcccccaagatgtgataaaattattagaattagaactgggggattcattacattggattgtaaatattgatgaaggaataactgtatctattgaaaaagctgaaaaataatcagttaatccttcacaaatatttttagtaaatattaaatatctttttttataataaatagtattggttactaaaatttttatcaactcgtggtatctattttttgtagcctctagtttttgagaggcgagatgttgccgcatcacagaccccctcatcaactaggaattacatttacttattttgcatttaattgttctttttcaacctttttttgttctgataacatcataaaatatgtaaagtataatctatttacaaaaccatattccccactattttctctcccaatagactctataatcccaaggtctttagctctctttaaaaaatcattaaaagctctcttctccccatttgtcaaaatatttttcacatctgattttttaaaatccattaatctattttcccctaatttaagaaaaatatcaatatatgtatcgcttctaattttatttaatttggattttaattgttttttagctaattcatctgttgcatcagtaataccagaaaatactatagattcatttatttttaaattatcttgagcattccaaaaaattgaatcaccaatttgttgcattattaaaggcataccccatgaaaaataaaccataggttctatagaattatatccatcttcaaactcaatgcctacattattaaatgaagttgtgaagaaatcttcaatatcattatcctctaaattatcaatttcaattaaattaaacattctagaaaatgattcattaattgaacataatttttcaaattcatgagggtaacttattaatgtgaaaacaacaggcatatgatattcattaaccaataatgtttcagataatcctttataccaatctgtaaactcttcattatcagataaaccattaagatcatcaacaacaataaatattccataatcttcaggtaattctttacaagtagtaatcaaaaaatctgcaaaatgttttttaatattatgaattaaattagtatcattatcttttaaagatactccagtccctgcaactttaaattcattaatattattcaaaattttatcaattacacttttacctaaataatcctttttaaattcttttaataatccatcaagtaacttttgtattaattcatcaatagtttttccaccaccattgttaatatggattggaatcatttgaaaatcatcttctgcttttcttgaaacataatttataaatgatgttttacccattcctctctttccagttatgaaaaaatgttcgggagctccttgatttttaactttaggtaaatattttataattttgttgatatcttcttttcttcctttaaaattatctggagatactggtctccctggttcaaaaggactttctgggaaatcttgtgacatgatatttttacccctcaatactcatttattacaatttacattttgtatttaactgtcttatcaaatttttattactgtatcagctagaaaatctggtgtaacattccgtttctttttttgatttaaaatagtattggaccatttactgaactgtgtagcttctttactattgacgcgatagcctacggaaatcatagcatctaaattatagagtatgggttgagtatttgcttcaggattaattttaataacgattccactaatagtggaatcgtttggattaacagttacttcatctttaacaagttctccactatcaaagatattatttaaatgtttactaatgttttttggtgttttattaaataaattagccattgttttttgtgttgcccataatgtttcgttttcttgatcaatgataactttaattgatactggcccatcttcacttttatataaaaatgtttcaataagattactttccaccatgatttatataacctctacaattttactcttcaaaaaagtttttttattcttgtttttatatttatgttataagttaatataaatgtttctattcaaaaaataaaccttaaaaatacagtatattcaatgaaaataatgaaacactaaacattattcagtatgaaaaaatgaaacgcattgtgttctaaattttggtgtttaattagttttgtgaactgttaggttattgttcatttttataatgaatattttttatctttttttgttattaaaatgtattgatatttttaattaaaaaaatagaaacctttatatactatgtagtacaatataataatagaaatacatggaggtgaaaagttgtcagaatcagaggcaataaaaatcataatattgtctttgagtataatcctagaaatactcaaaagacaaaaacacaagtaggaggttaaccccctcttacaattattatattttttattgcttcttatataaatttttcttaaaaaggaggtgaaaaacaaaatgacaataacaagcatcataatactaatattattaatcatattagcaatattaatctacattaatagaaaccaaaaatatacttggataagtttaataatatggttggttattttaataagtttcatctacgaaacattaatctaacccccccatattttttttatattataagccaaaaaaaaggaagatgataaaaaatgactgactacaccagcataagaataaaaaaagaaatagctgaaaaaatacaattaataaaaatacaaaacaactgcaaatcactaaacgaaacactagaacaactaataccccgaacagtaaacgaaaactacgagttcataaaagaacaaccaatattcacaataaacaacaaacccataacattcacagacctgaaaaacaacaatacaggcaaaacatggggaaatgaaaaacaaaacgcaacaatagtatttaaagataaacaaggagcatttataagattcaatgatgaagatgaagtattcttagaatactaccacttcatctaaaaccattctttttttaatattaaaaataaatccataaaacctcaaatttttaaagttttaaaaatttgttccataaggtacatagcatcatccaattcaccttcagagatattataaaaaggatcataatctgctttttttcttaaattaaataaatcatatagtttattcctaacagaatactgattgacctcaagaagttcatattgaacttgcccatgttctgtaataaactcattttcaccaaagtcttctttagacaatggtttaaatttataaacttcctgaagccacaaactaacatataaataagcactgtaatagcacctattgataatagtactaaacacacaacgattatcgcaattttcagggattaattcttctttattttcatataactgcttagtgaatttatataattcatattgagggtatttttcaatattaatgtataaaacccccaataaattatttaattaaaatataggtatctttatagaaatcaaacatattattttcctttgaaaagataaccatatcattaataatttggtctaacagagaatatttttctttcctatcaatatgaaatggaacattaattgtaaatactttttcaggatatgatccatcatacactgtatcaatagatatattttttcctaatcctaaattgtcagaaatttttataagttcatcataaaattcagtgattttactttcaaaagactctgtggatgattttttaaaaaacttatttttaaaaaaatcccctacaatattagaaggtttattatatccataatttttatcaactgaatacaccatagacaccacctataaatgtttattctaaagtttttctaataatatccaatgtttcaggattattttgaacatcaataagaactgtattactttttatttcttcatcattcctataagataaagtaagaatatccttatttatttccccatcttttaaaacaaaagctgcatttataggtataacttcaagattttcaaagttttcttcaagtttttttaaaaaatcaacattaaaaacttctttaagggaaatcccctcttgaagcactcttaagcttcttgcatcctttttcatttaacttcctccaaaatagttatttaataaatatcttttattaaacatataaaccttgtcaaaaaatttttaaattaataaatcttcaaagtcattttctttgaaattttcttcttttttctcaatatttcgtaatctttcatcaatatcatagattaatttttcataatctttgattttttcatctttttcaacaattgtattttctaattttaaaaattctggagatttaacagtgatcttttcaacttctttactaattgataatgctggtaaatgttgaatatattcctgttttaaatcagcaggattggtcatgaaatatacttcatcagtactgttttttgatttgccttgcaggtcatttactttgtccagactcataccatcattatatagtgttgaggcgtggaattttctgagcatgtggctgcggaatctgttgtagtttcctgcttttcctaatcctaattcattgtttattttaatgaattgttggtttaggtagtcttcatgtattttgaatagttgggattctggtgttaaattttgtcttgatagtaagtgatgatttatagctgtgactgcttctggactgcaataggtaatataatatttctgtgttttttgccttaaaatattaaaagtagggacaacattatcaatattatttaacacatctatcatttccataatattatctgtattatggtattctttagttgcgttcatatagtccattacagttaaatttaatgtttctcttctggcacaacctgaactactcatgaataatataatagctttcatggtagaagtacatatattaactgcttcacgaataatttctttatcaggtaaatctttaaaactgatcggtttgggattattataactttttttatcaattcgtggcagatcatgtatttcaatttcaaaatatttatatacaacaagaactggtgtgaaagtatttgatacagtgttataataataattatccatcaaatacttgcgaaaatttaataatcttctttttaatgtacgatgtttccatcgtataccctgttcttcttctttttctgcttcttcgattaattcggctaatgataacttattcaattcacaatatttatttattgaatgtttgtaaatatgctgtgttgtttttttatgatttttaacagaatggatctcatttaggatttcttcattggttctcatatttatcaaatctatttttctctttttatattgtttgatgtagagtgcatttgataagtatgtgataatatgtctactgtcgtgtctacaacgagttaattataacatacttaaaacattctaatctattaaaatattatatcaaaaataggttttaaattttatcccccaaaattaaacccttatcctcgtactgcgtttataaaaaatggaagtaattaagataaatttctccgtattcatcttcaaatctgattaatgctccaaggttatctttatagattaatgttgctttttccccattactccattgtgtgcctacttccgattgttttaattcattccatgatacatttaaaactgtttttttgtttattagtgtgaatgctggttgttcttgtatgtattcttcaggtgttgatgttccatcaggtaataaatgttttactgtttcattaactgatttgtatccgttgatttcctgtatttcttttatcttgttaattaaatttacttcaagttttatagtacttctttccattatttaattcctcctttaaaaaaataataaaaaaattatagtctttatattaaatggattataatgatgactataatatttattataagcattattgcgtttcgtgtcctgttttcttcatttttcattgaatataataatactactgcaagtattattaaaattatttgaattaatgttaatactgtggttatcataattccccatcccttttatttaaatattagtaatgacatataatttattataaattattttaaagagggattaattttaatcccccattagggataatattattgatattatccccataaccatatttattagaaaatatatgtcatttttatcaatgtttttcacctccttgtagttctattattatattggttactatactatataaaggtttctattttttactacttatttttataaaaaaataaaagaattgttgataaaacaacaatcttattgatacaaatctgagaaaatccaagctggatcatacgctaaaatcgtaccattcatacaccaattacttgaaacactgtttccatctaaaactgctgcagggtcacgataaatccaagaacccccagtatacttagaatgtttcaatctgagccttacatgcccagtcccacttctacatcgtacatgaatgaattgtacattataacctaatgcaagtcctaaacggtaaaataattgacttgaatcagtacaattaactccttgcttattatatatcttattaatagtttcctgagtattatatttggagttataataatatccatatcctcttcctcggattttgttcaagcaagagtctatatctgttacaccaccaaatttgtcggtgaacctttttaatgtgatgtctgtggtgtcgctggttgttccgttgccatgtgggtcttcaagataaacaatagctggactaacaccatttaatacttcataagctgatacacgattagccatactaacataacaattcttataatactttgttccatcagtatcagacacataatttgggaggaaacttaaatctttattatcaaatattgcacattcaacaaactctgcaataagactgtcttgttgataattcgtacctctctctttaattgcttttataattgtgccataatcagctacacgattcatgtaactatacacttgtcttgtattaaactttttattattattcactaaccaataagcaattctattcatcaaataagtgtattttgtaaaactaatatgttcattaaccatttattttcatctcctttttaaattttaaatcatctaaatattcctttaaaatactgcattttattattgcatcttcaaaacaacaaggaacctctttatcacaaggatcataaaatgcacatttttcacattcttcactaatcattccaaaataatcctcctatcaatttttaatgtacaatcagtaatcttgtttgttctaaacgaggattctctccagcagctcctctttttacaataccttccaacatataacttccagggtctaatctaatcggcagtattgctctaccattagcatcagttgttctttcataagaaacaccatttacaataaaattaattaccacattttcccctgtgttaatttcaggattggaagaaataattttaccgtaaacagaatttccataatttaattctagtttaactccatcttctccagcaaggtctgataagcgacccattactaatctagtgttttttaaatcatccattgtggtttctaatatttcttgtcttgcaaatgtttcagaatgttcatcatccaccaaatctgcatttaaacctgaacctgctccatccacttcttttaatttatttaaaatagtggtgggtgtttcaatgtttccatctttaccatcttttcctgcaggtccagtatcacctttacttcctttatcacctttaggacctgttggtcctgttggtccagtattgcctttagggcctttgaaattccccagtaatatttttttaaccattaaaaaaaacctcttcatttatttaattatactcctattttcattttcacaccagacacattataactcatacttgtaccgctactccatgcacctgcttttgtaattaatctccacctattaggattaatttctgctcctcgactcttattggctgtgttaattgtttgagaatcatcacataatacataacttaacttatcatgtcttactttgaaaattaaaccaaaatattttcctgcaacattcccatatgtgcccttttggtcgtaagttctatttgttccaccatcacaaactatatcaacccatgtacggccttgctgaggggtgcatactacacttattttttcgtttgtttctgggttcaataagataaattgtatattagagttagttcctaagctgttaggactccatgataaaaataagtcaaagttttcaagattaaaagtatctgtacgatacccttcttgagtgctctgcttgtagggtatttgtatggtatcgtgaacatgtgagtgagtaatacttttatctgcaatactgcttccttcaggcaatataacatcagataaaaagaaactaaaatctcttgtattaatattggttttaccatccaagcttgattgaagatcagtaatattgctgatactgtgactgtgactattgttagctttaccgttcaacgcattagtcacgattttattctgtacaggattaatagaagaactacttaatgatgaatcaacaactgttttattagcaccatcatcaatattttccaattttttgtcatgatcataaacaaaaccatttacacttgactgtctattaattccatattcatacacccacatgccatcatcactatcctgatcaaggtctatactatcacttgtatggctgtggttaacatttgctttatttgctaattgagtattcatttctgtttgtgtaacataccctgataagtcaactgttgtttcagttgaaccaattttttcaaatttattattaacccaaatatactcatcataaatattaccctcaacagaagctgaagtatttaattttaaatacattactccaacttcccctgtagatggtaatgaatttactattgttgctgtaaaaccaattaatgaatttatctgagaattaatattatttactttcgcatctatttcagtttcagtataatacctttcatcatgattatgtccaacatttgattttccacctaattttgaatcaatttctgttttcttataataatcatttaaatgtttaacagcaatataattgccaacatgtacctgataactatttgctccatcttcactagctatatttaaatataaatcaccagtagtttcatcatgttcaaatattggagggttggtttcatcattacaataacaccatagatttccttcagcatctcctgctaaagtgaaaaaacctggtggaggtactgtaatatttaataatgggtttgcccatttgtcgaacaattcatcaatggctttacttactgtattggcatctgtctttaatatgtcgatgtctaggaaatcttcaatgtttcctatttttgtgcctaggtttgtttctcttaatcttgcacatgcattatttaatgcttttctttcattttcagttagtttagtcatattacactctctcgtttaacattacctattttaatctctttgtcaccatttagataaggattataacttatttctgttatttgtgctttgaacacctctgaatctgggaatcttacatatac

(19) United States
(12) Patent Application Publication
(10) Pub. No.: US 2024/0074458 A1
(43) Pub. Date: Mar. 7, 2024

(54) AGRICULTURAL COMPOSITIONS AND METHODS

(71) Applicant: Carus Animal Health Limited, Reigate (GB)

(72) Inventors: Gregory James FIRTH, Sheffield (GB); Rajesh Muru ODEDRA, Stevenage (GB)

(21) Appl. No.: 18/492,384

(22) Filed: Oct. 23, 2023

Related U.S. Application Data
(63) Continuation of application No. 18/352,760, filed on Jul. 14, 2023.

(30) Foreign Application Priority Data
Jul. 15, 2022 (GB) 2210434.3

(51) Int. Cl. A23K 10/18; A23K 20/105; A23K 20/147; A23K 20/158; A23K 50/10

(52) U.S. Cl. CPC A23K 10/18; A23K 20/105; A23K 20/147; A23K 20/158; A23K 50/10

(57) ABSTRACT

The present disclosure relates to the reduction of methane production in ruminants. In particular, the disclosure relates to solid and semi-solid compositions for administration to a ruminant comprising a substance capable of inhibiting the production of methane in vivo in the ruminant. The disclosure also relates to controlled release boluses, animal licks, and animal feeds comprising solid and semi-solid compositions comprising a substance capable of inhibiting the production of methane in vivo in a ruminant. The disclosure further relates to methods of orally administering boluses capable of inhibiting the production of methane in vivo in a ruminant to ruminants and methods of orally transferring substances capable of inhibiting the production of methane in vivo in a ruminant to ruminants. In addition, the disclosure relates to viruses capable of inhibiting archaea that produce methane in vivo in a ruminant.

### SEQUENCE LISTING

The instant application contains a Sequence Listing which has been submitted electronically in ST.26 XML format and is hereby incorporated by reference in its entirety. Said ST.26 XML copy, created on Jul. 11, 2023, is named 16408.0001-01000 Sequence listing.xml and is 40,561 bytes in size.

### FIELD

The present disclosure relates to the reduction of methane production in ruminants. In particular, the disclosure relates to solid and semi-solid compositions for administration to a ruminant comprising a substance capable of inhibiting the production of methane in vivo in the ruminant. The disclosure also relates to controlled release boluses, animal licks and animal feeds comprising solid and semi-solid compositions comprising a substance capable of inhibiting the production of methane in vivo in a ruminant. The disclosure further relates to methods of orally administering boluses capable of inhibiting the production of methane in vivo in a ruminant to ruminants and methods of orally transferring substances capable of inhibiting the production of methane in vivo in a ruminant to ruminants. In addition, the disclosure relates to viruses capable of inhibiting archaea that produce methane in vivo in a ruminant.

### BACKGROUND

Methane is produced as a by-product of the digestive process in ruminants. The digestive process involves enteric fermentation of cellulose in the rumen so that the ruminants can derive nutritional benefit from foods such as grasses. Ruminant production accounts for about 81% of greenhouse gases from the livestock sector, 90% of which results from rumen microbial methanogenesis. Enteric fermentation carried out by microorganisms produces a range of metabolites that are utilised by the host animal as well as waste products, including hydrogen. Hydrogen in turn is utilised by other microorganisms, including methanogenic archaea (which are commonly referred to as methanogens). Exemplary methanogens found in ruminants include Methanobrevibacter millerae, Methanobrevibacter thaueri, Methanobrevibacter ruminantium, Methanobrevibacter smithii, Methanobrevibacter gottschalkii, Methanosarcina barkeri, Methanomicrobium mobile, Methanobacterium formicicum and Methanobacterium bryantii. When methanogens metabolise hydrogen in the enteric fermentation process in the rumen, the predominant biproduct is methane.

It is well known that the livestock industry contributes significantly to global greenhouse gas emissions. The emission of methane from ruminants is a particularly prominent contributor to these greenhouse gas emissions. Global meat consumption is rising and the livestock industry is expanding to fulfil demand. Ruminal methane emission, therefore, and the environmental impact of livestock production is expected to continue to rise in the future.

A number of strategies have been considered to aid the mitigation of methane production including feeding extracts of plants including garlic and red seaweed, which are substances that are reported to reduce methane production. In addition, specific chemicals including anti-protozoal agents, aliphatic bromine compounds present in red seaweed extract, oils from garlic extract and other plant derived chemicals have been considered as possible interventions in mitigating methane production.

There is a need for new strategies to minimise the environmental impacts of the livestock industry, and in particular to minimise the methane emissions associated with ruminants.

### BRIEF DESCRIPTION OF THE FIGURES

FIG. 1 shows a side view of a bolus arrangement according to an embodiment of the disclosure.

FIG. 2a shows a perspective view of a tubular bolus.

FIG. 2b shows an end on view of a tubular bolus.

FIG. 2c shows a schematic representation of the cross section of a tubular bolus when it contains concentric laminations of different substances.

FIG. 3 shows transmission electron microscopy images of a culture containing Methanobacterium bryantii and virus isolate 1 of the disclosure after four passages.

FIG. 4 shows transmission electron microscopy images of a culture containing Methanobrevibacter smithii and virus isolate 2 of the disclosure after six passages.

FIG. 5 shows the gas chromatography mass spectrometry (GC-MS) relative abundance for methane production by Methanobacterium bryantii for an ex vivo culture including Methanobacterium bryantii and rumen virus isolate 1 of the disclosure, compared to a culture including Methanobacterium bryantii and with heat-treated rumen virus isolate 1 and Methanobacterium bryantii with no virus present after 48 hours of incubation.

FIG. 6 shows the gas chromatography mass spectrometry (GC-MS) relative abundance for methane production by Methanobrevibacter smithii for a heat-treated artificial rumen containing Methanobrevibacter smithii and rumen virus isolate 2 of the disclosure compared to a heat-treated artificial rumen containing Methanobrevibacter smithii with no virus present and a heat-treated artificial rumen with no microorganisms present, each after 48 hours of incubation.

### SUMMARY

In a first aspect of the disclosure, there is provided a solid or semi-solid composition for administration to a ruminant, wherein the composition comprises a virus capable of inhibiting the production of methane in vivo in the ruminant. There is also provided controlled release boluses, solid or semi-solid animal lick blocks and animal feeds comprising a composition comprising a virus capable of inhibiting the production of methane in vivo in a ruminant. The semi-solid composition or animal lick block may be in the form of a colloid or a gel.

In a second aspect of the disclosure, there is provided a method of reducing the production of methane in a ruminant comprising orally administering a bolus to the ruminant, wherein the bolus comprises a virus capable of inhibiting the production of methane in vivo in the ruminant and provides controlled release of the virus to the ruminant.

In a third aspect of the disclosure, there is provided a method of reducing the production of methane in a ruminant, comprising providing an animal lick block or animal feed which includes a virus capable of inhibiting the production of methane in vivo in the ruminant, wherein the virus is orally transferred to the ruminant when the ruminant consumes and digests the components of the lick block or animal feed.

In a fourth aspect of the disclosure, there is provided a virus capable of inhibiting the production of methane in vivo in the ruminant, wherein the virus targets archaea which are involved in the production of methane in ruminants.

The compositions and methods of the disclosure provide an effective and easily controlled means to reduce the methane production from a ruminant, and thus for reducing the greenhouse gas emissions from the livestock industry.

### Definitions

The term “comprising”, “including” and “includes” as used herein means that at least all of the listed elements must be present, but other elements that are not mentioned may also be present.

The term “consisting of” as used herein means that only the listed elements must be present, such that other elements that are not mentioned are not present.

The term “semi-solid” as used herein to describe the form of a composition refers to the composition being in a form having a viscosity and rigidity intermediate between the solid and liquid forms of the composition. Exemplary semi-solid forms include but are not limited to gels, colloidal forms, creams, and suspensions.

The terms “lick block”, “lick”, “animal lick” and “animal lick block” are used interchangeably herein to refer to a source of feeding supplements for an animal, which is adapted to transfer the feeding supplements to the animal when the animal licks it. The feeding supplements may include minerals and nutrients.

The term “virus” according to the present disclosure includes double-stranded or single-stranded RNA or DNA viruses, which infect cells of bacteria archaea, plants, and/or animals. As used herein, the term “virus” in the singular can refer to one or more viruses. Thus, the term “virus” in the singular encompasses both the singular and the plural, i.e., it can refer to one virus or multiple viruses.

The term “engineered virus” as used herein refers to a virus that has been modified from its naturally occurring state to have enhanced activity or to be stabilized by covalent linking to a substrate.

The term “archaea” as used herein refers to any single-celled prokaryotic organism.

The term “substrate” according to the present disclosure is understood to mean any solid phase material to which a virus may be immobilised.

In the context of the present disclosure, “release period” means the amount of time for which the active substance is released to the ruminant.

The terms “substance capable of inhibiting the production of methane” and “active substance” are used interchangeably herein to refer to a substance capable of inhibiting the production of methane in vivo in a ruminant. Thus, the term “virus capable of inhibiting the production of methane” refers to a virus capable of inhibiting the production of methane in vivo in a ruminant.

The term “inactive substance” herein means a substance which is not capable of inhibiting the production of methane.

The term “non-virus containing substance” herein means a substance which does not include a virus. The non-virus containing substance may optionally be a substance which is commonly administered to ruminants, such as minerals and/or anthelmintics.

As used herein, “controlled release” means that the active substance is released at a predetermined rate or at predetermined intervals. Controlled release includes sustained release and intermittent release.

As used herein, “sustained release” means that release of the active substance is maintained over the full duration of the release period. Sustained release encompasses embodiments wherein the active substance is released in a constant dose across the full release period and also embodiments wherein the active substance is released at varying doses across the release period.

As used herein, “intermittent release” means that release of the active substance is discontinuous, such that there are intervals during the release period where the active substance is not being released.

As used herein, “majority” means more than 50%. In some embodiments, this term means 60% or more. In some embodiments, this term means 70% or more, 80% or more, 90% or more, or 95% or more.

As used herein, the term “about,” as applied to one or more values of interest, refers to a value that is similar to a stated reference value. In some embodiments, the term “about” refers to a range of values that fall within 25% of the reference value in either direction (greater than or less than the reference value). In some embodiments, the term “about” refers to values that fall within 20%, 19%, 18%, 17%, 16%, 15%, 14%, 13%, 12%, 11%, 10%, 9%, 8%, 7%, 6%, 5%, 4%, 3%, 2%, 1%, or less of the stated reference value (except where such number would exceed 100% of a possible value).

### DETAILED DESCRIPTION

The present disclosure relates to a solid or semi-solid composition for administration to a ruminant, comprising a virus capable of inhibiting the production of methane in vivo in the ruminant.

The disclosure also relates to a method of inhibiting the production of methane in a ruminant comprising orally administering a bolus to the ruminant, wherein the bolus comprises an effective amount of a virus capable of inhibiting the production of methane in vivo in the ruminant and provides controlled release of the virus to the ruminant. The disclosure further relates to a method of reducing the production of methane in a ruminant, comprising providing an animal lick block or animal feed which includes an effective amount of a virus capable of inhibiting the production of methane in vivo in the ruminant, wherein the virus is orally transferred to the ruminant when the ruminant consumes and digests the components of the lick block or animal feed.

The disclosure also relates to controlled release, solid or semi-solid animal lick blocks and animal feeds comprising the compositions of the disclosure. In some embodiments where an animal lick block is provided, the virus capable of inhibiting the production of methane is covalently linked to at least some components of the animal lick block.

The disclosure also relates to a virus capable of inhibiting the production of methane in vivo in the ruminant, wherein the virus targets archaea which are involved in the production of methane in ruminants. In some embodiments, the virus is an engineered virus which has been prepared for the disclosed methods, compositions, kits, and uses. In some embodiments, the engineered virus has been modified from its naturally occurring state to have enhanced activity. In some embodiments, the engineered virus has been modified to be stabilized by covalent linking to a substrate. In some embodiments, the engineered virus has been modified from its naturally occurring state to have enhanced activity and to be stabilized by covalent linking to a substrate.

Viruses of the present disclosure may be engineered to provide enhanced activity and performance. Routine modification of natural viruses using CRISPR and Cas9 can be carried out to eliminate undesirable traits such as lysogeny. Examples include the elimination of the integrase gene within the virus characterised in the Examples by SEQ ID NO:1.

The present disclosure offers a means to inhibit methane emissions from ruminants, and thus to reduce greenhouse gas emissions from the livestock industry. The ruminant may be a bovine, caprine, elaphine, or ovine animal, including a calf or a lamb. In some embodiments, the ruminant is a bovine animal.

The virus capable of inhibiting the production of methane in vivo in the ruminant may interrupt the metabolic pathway of methane production in the ruminant, and/or may be capable of disrupting the enzymes of gut microbes in the rumen. For example, the virus may either be involved in the metabolic pathway of methane in the rumen, or alternatively may be capable of disrupting the enzymes of gut microbes that live in the rumen. In some embodiments, the virus is an inhibitor of methanogens. The virus may directly inhibit growth of methanogens or indirectly inhibit growth of methanogens.

In some embodiments, the virus is an inhibitor of hydrogen production that fuels methanogenesis. The virus may be a virus which directly inhibits the growth of hydrogen-producing archaea or bacteria. Alternatively, the virus may be a virus which indirectly inhibits the growth of hydrogen-producing archaea or bacteria.

In some embodiments, the composition comprises a further active substance which is capable of inhibiting the production of methane in vivo in a ruminant, selected from a brominated aliphatic compound, a carbohydrate, an ester, a lipid, a peptide, an enzyme, a further virus, a phlorotannin, or a combination thereof. In some embodiments, the composition comprises a further active substance which is capable of inhibiting the production of methane in vivo in a ruminant, selected from a bromoform, 3-nitrooxypropanol or hydroxypropionic acid, or a combination thereof.

In some embodiments, the virus capable of inhibiting the production of methane in vivo in the ruminant is a stabilised virus. For example, the virus is stabilised as described in EP 1496919 B1, by immobilisation to a substrate. This is advantageous because it ensures that the virus retains its ability to inhibit the production of methane while also improving its stability relative to a free virus.

Stabilisation of the virus may be carried out by attachment of the virus to the substrate via covalent bonds formed between the substrate and the virus. In some embodiments, the virus is immobilised via its head, leaving the tail free. This ensures that the virus retains its activity, because the tail of the virus is involved in its ability to recognise and infect specific bacteria and/or archaea. In some embodiments, the virus may be a bacteriophage. Suitable substrates include silica, chitosan or animal or vegetable protein. Such stabilisation is carried out to provide enhanced stability of the virus. An example where a virus is stabilised by immobilisation to a substrate via covalent bonds formed between the substrate and the virus is described in EP 1496919 B1.

Advantageously, in ruminants, where methane production in the rumen is caused by specific methanogens, the use of viruses offers a particularly effective means to provide targeted disruption of the methane production process. Unlike other methane-inhibiting agents, which provide a less targeted approach and can cause toxicity to the rumen, viruses are specific to their target bacteria and/or archaea and thus do not have problematic side effects for the ruminant.

In some embodiments, the virus is a bacteriophage which targets bacteria in the rumen, such as bacteria that produce hydrogen during fermentation that acts as fuel by methanogenic archaea. This is advantageous because the inhibition of these bacteria will, by extension, negatively impact the ability of methanogens to produce methane in the rumen.

In some embodiments, the virus is a virus which targets archaea which are involved in the production of methane in ruminants. In some embodiments, the virus is a virus which targets metabolic processes leading methanogenesis. It has previously been observed that the mix of archaeal populations vary between naturally low methane emitting cattle compared to animals that are high emitters. The use of viruses which target specific archaea is therefore advantageous because it allows the targeting of archaea which are found in high emitting ruminants without perturbing the other members of the ruminant microbiome. Further, the use of such viruses in the controlled release compositions of the present disclosure allows methanogenic microorganisms to be specifically targeted throughout the life of the ruminant. As such this provides a means of delivering the active agent without further human intervention.

It will be appreciated that viruses capable of inhibiting the production of methane in vivo in the ruminant can be isolated by the skilled person. For example, to isolate a virus which targets archaea, archaea cultures can be prepared in suitable growth media such as 119 Broth. Then, liquid can be harvested from a suitable source of viruses, for example, bovine rumen fluid, rumen faecal material, municipal sewage systems, or environmental samples from sources that provide habitats for Archaea, such as anaerobic soils including peat and peat bogs, thermal springs, and deep ocean thermal vents. Viruses can then be separated from the liquid, concentrated, and purified, for example by PEG precipitation and ultracentrifugation. The archaea cultures can then be inoculated with the resulting virus suspension and the cultures allowed to grow to allow viruses with specificity for the archaea to initiate a cycle of infection and amplification. These viruses can be filtered from the cultures and passaged repeatedly (i.e., repeating the previous steps with fresh archaea) to obtain pure active viruses. Similar procedures can be followed to isolate other viruses, such as those which are active against other bacteria in the rumen.

Following a procedure as described above, a novel virus with activity against Methanobrevibacter smithii was isolated, which had a sequence as set forth in SEQ ID NO:1. The isolation method is described in more detail in the Examples. In some embodiments, the nucleic acid of the virus capable of inhibiting methane production in vivo in a ruminant comprises at least 50% identity to SEQ ID NO: 1. In some embodiments, the nucleic acid of the virus capable of inhibiting methane production in vivo in a ruminant comprises at least 60% identity to SEQ ID NO:1. In some embodiments, the nucleic acid of the virus capable of inhibiting methane production in vivo in a ruminant comprises at least 70% identity to SEQ ID NO:1. In some embodiments, the nucleic acid of the virus capable of inhibiting methane production in vivo in a ruminant comprises at least 80% identity to SEQ ID NO:1. In some embodiments, the virus capable of inhibiting methane production in vivo in a ruminant has a nucleic acid comprising at least 90% identity to SEQ ID NO:1. In some embodiments, the virus capable of inhibiting methane production in vivo in a ruminant has a nucleic acid comprising at least 95% identity to SEQ ID NO:1. In some embodiments, the virus has a sequence as set forth in SEQ ID NO:1.

SEQ ID NO: 1:
GTCCTGGTGTTTCTCCAAGGTCTACTTTTATGCTTTCATTTTTCTCTAACCATTTTGTGT
TATCTCCCGAACCGTTTAATTTTTGCCAGCACCCAATCAATACTTCTTCTACTGATTGTT
CGCTAGTTTCAAATGGTTCTAATTTTGGATTACAATCACATACTGTGAGATAATTGAAAT
CTACAAGTGTTGGTAAAGCATGAATATATAATTCTCCTCTTGGTTTTACATAGGGGAATC
TTGAAAATTCAAAAAGTTTTGTTGTATCACATGTTAATGATACAAAATCACATCCTTTTA
GTTTGAAAGTTACGTTGCCGTACCAGTAGTTGAATTTTGATTGTTTTGTGATTACATTTA
CTATATTTTGTTTTGAATCACTTTCATCACCAGTATATTCTGCAGTTATTTCTTTATATT
CTCCTACTTTTTCTTTGACTGTTTTTGTTGCAGTGGTTGTTGTTTCTGCTTCTCCAGGAG
TTAATTTAACTTTTTTACATTTACCATGACCCCATTTGTCTCCTCCACAGTATCCGCAGT
AATCTGCGTCACATCCTCCTTTTTTCATGCTGCAAGTTATTTCGCCTTCGTACACTTTTT
TAGGATTGTCGGTTAATGTTCCAGTTTTGCCACAGTTTGGGCAGTAGTTTTTCCAGGTTT
TTGTGTATCTTTTATATGCTGTTCCTGCACAACAGCCACATGATGGCATCATGTTTACGG
TTATTGTGTTGGGATCTCCTGTTGTGACATTTTCAGTTACCTGTTTTTCTACTTCTTTTT
CAACATATTTCATGAATTTTATTGACTTATTTTTGTAGTCAATAGTTATTTCGCTTTCAT
CCTGAATTAGATTCGGTTGTTGGAAATATATCATTGAAGTATTGGGATCATCATCTGCTA
TTCCCTGATTAGTTATTTCAATTCCTAGTGTGGATGCTCCTTTTATTTTTTTAATTTTGA
GAATAGGGTATGATTCGCTGATATATTTTTCAACATCTAAAATAGTACATCCTGGGATTT
GACGTGGTGTAATGGTTGGGATTTTATTTGAAGAGGTTGTATTTTGTGTGTACATGAATG
GCATGTATTCGTAGTTTTCATAATCTTTTATATAGTGAATATTTTCTTGTACAAATGTTA
AAGAAACAGTGTCTATTATTTTTAGTTCATTAGAATTAAAGAAAACATCTTTTTTTGTTG
TTAAATATTTTGCAAGAACATATATCAAATATTGGAAGCTTAGTCTTCCACCTGTAATTG
ATAAGCTGTTTGGTGCTTTTGTTTGAGTTATGCAGTACGATTTGATTATTCTTGCTAATT
GTATTATTTCATCCTGTTTTATGATGCAGTCAATATGGCTGTGTGATGGTAAATCTGCAC
AATCTACAATTTTAAGTGAAATGTTTTCAGAGTTATTGTCTTCAGTGTTGATTATTGCTT
CTGCAAGTAGAAGTATCATTTGTCCCATGCCTATTTTAACATTGTAGACTTTGTTTTTTG
ATGAATCTTCATATATTTCACTATGTGATATTTCAAACTTGTTTGGGAAGTATTTTTGAA
CATCAAGAAGTACTAATAGTTTTTCAGCACAATCAATAATGTCTTGTATGTAAAAATCAT
ATTCAAATAATGTTGGTGGAATATTATTGTTTACGTCAAGATTGTAAAATTGTCTAAGTA
TTTTTGAATAATCTACTTCATCAGTATTTTCTGAAGAAATTACTGGCATTATACCTAATG
CATTTTTGGTTTCATCACTACTATATGATAATTCATTAGTATTTTCTCCAATTACAACAC
TTTCTACAATTTTATTGTGTGTTACTCCATAATTTGCAGGTTTTAGTAATTGTATTTCTT
TTATAATGTTATTATTTTTTAAAGAATAATTTGTTTTAAATACTAATCCTGATTGTTTTT
CAATAGCTTTTAATAAGTTGTATTTTGTTATTGAGCCTTGAACATTGATTAATCTTTTAC
TATAATCAATACTGTCAATATCTGTTTTTGTAACATTGAAAAATCCTTTCAGTAATTTGT
TTAGGAATAATTTACTGATAATGATTGTATTTCCATTTACATGTGAAGCATAATTGGGAT
CTCGGATATAGAATGGTTTACAATTGTTTAATTCTACAATTATTTCTTCTGCATCTACAT
CAATAGATTTGTCTATATAATCAAAGGTGCATTCACTATTAATAACAAACAAACAATTAT
CAACAAAGATTTTGTTTCCTTGTTTGAATAATTGCTTGTCTCTTTTAACATTTGTAAGTT
TATGTGATAGTTTTAATGTTTCATATCCTTGATATTCATCAGTAATATGTATTTCTGATA
ATTCAGTATCTAAAAATTGAAGAATATTTTCTTGATTATCTAAAACAACAACCATACTTT
TTATCCTCCTTTTTCATAGTAAATTACATCAGTCACTTTACAATTGATACTGTTTTCAAA
ATTAAAATGATGATCCAATATGAAAAAACTAGAATCTACACTAATACAATTAGGATCTAT
CTTGAACCATTCACTATATTTTTCATAGAATACATTTCTATTTTCACAATCAATTTTAAT
CTTTGTTGTTTCAGGTAAGTTTGTTATAAACTCTCCTTCAAGTTTCATTAGTTGTCCAGA
TTCTGATTCAATTATTTGTATGCTTGTTGATTCATCTTCACTTAATTTATACAAAATAAT
GTCTGGTTTTACTTTTCCAATTGTGGTTATTGTTCCTGCGAAACTTTTTCTTAATGGGTT
AATATTTCTACTTAAACCACTAGGGATTATTAAGTTCACTTCACAATCATAACCTCCAAC
CATTGCTTCTGCATCAATTGAATCTTCTATATAGTAATCAAAACATTCTTCAGGTGCGAA
AAAGAAACTAATACTTTTTAGAATTGGTGAATCTAATGTGTCTCTTTCAGGATACAGGTA
GTCACTTACATATTTCATGATTTCTGTTGATTCTTCAAAATCATCTCCGTAAACTCTGAA
TTTTAATTTGATTTTTTTATCATCAACATTGACTCTTATAGGGTATTTCCCATCTGCTCC
TTCAATTTTATATTTTTCTGTATCAAAATTAGCTCCACGAGGTATTTCTGTTTCAGGACT
CATACTTATAAGGAAATATTTGCAACTAACACCATTAATATAGAAATCGCAGTCTTCTCC
ACTATTTTTTGAATAATATACTGTGATTTGCACATTTTTCATTTCGACTTGGACTTGAGT
GACATTGTCAAATGCATCATCAATTTGTAAGAAAAACCTTAAATCATTTAATATTGTGTT
GATTTCTGGAAATGGAATGCCGAAATTATGGAATTTTCCACCGAATTTAAAGTTAGTGTC
ATTTCTAGTGATGTTTATGCTGTCTAAATAGTAATCTAATTCATCATCATCTAATGTTCC
AACACCACAAATTAGATTAATTGATTCTTCAACACTTATGTCTCCAGTTATTTCTATTCC
ATGAATCAGTACATTATTGTTTTCTAATCCTTGCCAGTTGAAACCTTCAAAGAAATGTTT
TGTAGATAAAGCTAACTCTGGAGGTTCAAGTAATAAATTTGCAGTGCTGCCTACATCACC
TAACAAATATCTTTTTGGAGATAAAGCCAATACTGGATATTCATAACCTGCATATCTTTC
TTTAAGTATTAAAGATAAATTACCAAATTCTACAGTTCCATAGTCAAAATCTATGAAATT
CCCATAAAAAATTAGATAGATTGGTTTACTTGCATCATATGTAAACTCACAGGATAACTC
TTCCCATATGTCCTTAACACTGATTTGATTGGTGTATGAATTTGTTGTTCCATTTACAAC
ACAAACTCTTAAGTTTTTCAAACCTTTTTCAATTTTAGAAGCATTTAACAGTCTTCCTAA
TGCTGAGAAAACATAAGTTTCACCATGTTCAAGGTTTGATAATGTGAATTCTGATAAATT
CAATATTGAATAATAGGATTTATATGAATCAGGAATGTTTACTTTCACTTTATCTGTGAA
AACACCTAATAATAATGGACTGGTCATTTCAATATTATGCTCTCCACTACTTTTAGAGTA
TAAAGGAATATTTGTATCAAAAACAATGTTTCCACTATCATCTTTTTGCCATTGTGATTT
TGAAATTTGTAAAATATTATTTGATAATCCTGGAACAGGTTCTTTTACAGTTAATCCTCC
AAAATTCAGATTTAGTGTTAAATCTAATGGAGCATCAGTTTTTAAAGTTATGTTTAAGTA
TGTTAAATCTGTAACTGTATCTGTACTAATTTCAATTTCTTTGTTATTTGCATAGAAATC
CCATACTTCCAATTGGGGTTTTTCTACTACAAATGTTGTTTTTGTTGAATGGCCACTATT
GTTTTCATCAAGTGTGATTGTAAACAATCCTTCAGATGTTGGAGTTACATAGAATATTAA
TTCTGCTTTACCATTTGTTGTTGCTTGTCTCCATATGCCTGTGGTCCAGTCATAATATGG
TGAATCATATGTGATTTCACAGTCTCCATTTGTGATTATCTTTATGTTTGCCGATATTTC
TTCTGAAGGTTTTGTTCCACTGGTTTCAACCATTGTGCATTTTACAGCTACTTTTTCACC
GTAACTAACAATATTTGGTTCAATGCTAATGCTTGGAATATAATTTCCAGTTGTATACAC
TACAATTACTCTTAAGTATTTCATTTCAATGTAACATGGGTTTGTAGATGTATTTTGTGG
TAAATCAAATTTAACATCAAAATTTGGAGTGTTAATGTCTTTTCCTTTTAGATTCAAATT
ATTAAATTGAACTTCATATTCTCCAAAATTATTTCTAGGTGGGGCATTACCTGTTTTTGA
CAGGTTTAAACTACTTAATATGATTTCTGGATTTGAAAAACTTCCATGTGCAGTTTGACT
TATCTGTGCAGAGTATGCTAATTTTCTATATGAATATTGTACTATTATACTCGTTACTTT
AGCTGAATCTGGAATTAATCCATCATATTTAAAATCATATCCACGTATGGTTGATGGATG
TTTTCTTGAACCATTTCTTCCAGCAATCGCATTTTTATATTTTGCTCCAGGATCTGCATA
ACCCCAATGTCCCACAGTATTTAAATCATTTGTAATATTTTCAATATGATCACATGAACG
ATAAGGTATGTTGTTTCCTTCTATATTTTGAACTTTACTAGGATATCTTAAAATACTAGG
CAATTTAATAATCCCTCCATCATTTTTTTTAATTTTTAATAAAACCATCATACTCTGAAT
TAAACAATAAACAGTTTAATTGAAAGCTAATGTAATCTCCTTTACTTACCCCATCAGGAA
AAGCAAATTCAACACTCACATATTTAGCACCAGCAGGTATTTTTGATGTTTTAATAGCAC
ATCTTCCTTTATTACCCATACCTTCATTAGCAATATCCATAATTTCCTGATTTTCAATTA
ATTTACCATCTTTATCGAAAAATACAAACCCAACAAGTATGCTTTCAATAGTGTTTTTTG
CATCATAATTAGATGAAAACACAATACTGGAAGCATCATCAGGTATGTGTACTCCTTGAT
TGTTCTGATGGATTAAAAGAAAAAAAGGAGAAGTTAAATCAACATTCTCCGAGAGATATG
TTTCTTTAACTTTCAAAGTATAACCATTTCTAAAAGGAACAACTTCAAAAAAATCACTAT
ATGGGTTGAAATTAAAATCAAAATGTTCACAAACAAACAAACTAGATAAACTAGATTCCA
TACAATATTGAATATCTTCACTAAATCTGTTAGTTAATGAAACATCTTTACTTAATTTAA
ATGAATATACATTGTAAGATTCCCATTCATCAGGTTTTACATTATTTAATGAAACCATAC
ATTTATGTGATTCAAAATCGTTTTTATCTTCCAGATATCCATGTTTGTTACTTATGATAT
TACCGAAGATTTCACATTCTTCAGGATTAATTTTTATAGTTTCCATTATCTTTTAACCTC
TCATTCTTTTAATACGATTGTCTCTTTTAATTAAAGCTTCAGCAATGCGGTCAATTAATT
TGGAATCAGTTATCACTTCTTTTAAAGTTGTGATTATTGATTTTTCATCAATATCTTCTC
CTTCAACAGTAACATTAAGATTATGATCTATTGTTAAATGCACATCATCAGATGTATAAG
ACACTAAATTATCAGCAGTAGTATTATTCCTATTATGTTCAATATTCTGGAAGTATGCAT
TTTGATTTGTTTTCATTAAATCAATAATATTATATATTCCATCTGTAATGATTCCGAATA
CATCTTTAGAACTTCCTCCAGGACTTGCTGAACCAAAACTAACTCCAGAACCTCCGCGGA
ATATTCCTCCACGTTTTTGGAATTGTGTCATATCATATATTTGACCATTAACCATAGCTG
CTACATGGCCTATTCCATTCCATGATCCATGAATCATGTGTCCTGATAATCCCAACATAG
ATGCTATTTCAATTATCATTTCTGCACCATCAAAACAGTTGCATCTTACTTGATCCCAAA
CTTGCTGATTACTATATCTTGAATTATAATAAAATTCATAAGTTCCAGGGTTTTGGAATC
CTCTTGCAGTTAAGATTTTTCTTAGAAGATTTTCAAAGTTTCCAGGATTTAATTGCGGTT
GTTTTCCATCACGGAAATCTTCCACATGATAATCTGAAGGTATTTGAATTCCAAGGAACC
ATGGGTCTGCTATCCTCCATTTGTATGCAGTATTCATTATTTTATTCACATTAGTATCAG
GAATTCCTGCAAAACAAGTATTTGGATTAACACAACCATTAACTCTTGCCAAATCATTAA
TTACGCTTGTTGGTAAATTATAATCTAAAAGATTTAACCATGATTTGCTTTTATTGTTTT
GAGGATGTTTTACTTTAGAATTAGTGTCAATTACTCTGCCTTTTCCTTGTCCTAATCCTC
CAGCAGCATAACTTGCTATTGTGCCTTTTGTGGATTTTCCAAAGTTAATGCTTCCACCAG
GTAATGTTCTGCCTCCAATTAATCCTCTTGTTGAATTAGTTGCTGGGCCTGCAGGCAGTC
CACTACTGAAATGTGCATTGGCTAATTGATTATAGAAACTTGCAATACTTCTATGAAGGC
TGCTGAATTTATTGTATGATTGTGTTTGTATGCTTCCTGCAGCTGAAACAATATTGTCTT
TCATTACTCCCCATGCATTAGTCATTTTATTTGTTACATCTATTGTGGAGTTACGTACTT
GATTTAAGTTCTGGGTTGTTGTTGATTTAATATTGTTCCATGCTTGAGTATTTGAATTAC
TCATGCTTGTTAATGCAGTGGTTACTCCATTACGTGTTGTTTGGAAGCTTGTTACTACTC
CTGCAACTCTCATTTGAATAGCTCCTGCATTTAATGCTATTCCTGCAGTTAATGTTGAAA
AGCTTGTTCCGATCATATTGTTGCTTGCAGTGATGCTTTCACTAGCTAATGATACTTCTT
CAACAAGACCATTATATTGGTCTCCAGTTTCTTCTGTACCTGAATTATTATTGCTTAAAT
CAACATTTGCACTATCTGTTTCAACTTCAGTGTTTGTTGTTACTGGAGTTGTGATAGTTG
TTGAAATATTATTTAATGCATTTTCAACATCAGCAGTATTGAATCCATTTACAATGTTTT
TTCCAACATTTTTACCTGCTTCATATGCTGATTTTCCTTTGTCTTTAACTCTTTGTAGCA
TGTTAAAGAATTCTAGTACTACTTTTTCTTGGATTATTCCTGGTGAATGTATTCCCATTG
CGGAAAGCATTCCGTCTACAATTTTTTTACCGATTTGCTTTGCATTTTCAACTAATTGTG
AACCTGCAGATAGTATTCTTGAGCCTATGTTTAGGAATTCCTGGTATACTTTTTGAGGTA
ATTGCTTTATCCAGGACATTACTCCAGATACTACTTGTGATGCTTTTGTTTTAGCATTAC
TTACCCATTGTGCTCCTGCAGTTATTACTCTTACAGCAACTGCTAAAAGATACATTTTTA
TTTTATCTGGCAATTGTCGGATAAATTGAACTACTCCTGTAAGGAAGTTTCTACCAGTAT
TAACTGCATTATTCCACATTGACTGAGCCCAACTAACAACACGATTAATCACATTTGATA
ATATTGTGCTGATTGTTGATTGAATAGCTGCCCAAGCCCAAGCAATTGCTTGTTGAAGAC
TTATTTGACCAGTAATTAACTGGCTAAAAATTGTTGCCAGTTGCATGAATAAATTGAATA
ATGGAGTTACAATATCAAGTACAAGTTGCCATGCTGGACCAAATACACTTAATAAGAATG
ATGCAATTCCATTGAAAACATCCCATATTATGGTGAGTATGTCTATTGCAAGTTGAACTC
TAACTTTGAAACTTTCAAATACTGCTCCTATTGCATCGATAACTGCTCTTGTTCCATCAA
CTTTACCTTTTGCACTTTCTGGGAATATTTCATCCCATATTCCTTTCAACCAATCTACAA
CTGGTTTGAGAGATTCATTCAAATCAGACCATGCATCTTGAATTGCTTTGATAGTTGCTT
TTACATCAGGATGATTTATGAATGCATCCCATAATCTACCAATATTATTCTTTATGGCTT
CGAGCATGGTTCCTACATCTTTCCACCAACCAAAAGCTTTACCTACTTCATATATTGCTA
CTGCTATTGCAACTAATGCCACAACAACCCATGTTAATGGATTTGATAAAATTGCTCCTG
CAACTGCTAAAAAACTAGCCGCCAAACCCATATTTGCTATTTCAACACCTAATGCTCCAG
CAATATAAGTTACAAAACTAGTCGCACTAGCAGCTATTGCGGGAATTACTGAACCAAGAA
TTACAATACCCAACCCAACTAAAGCTGTTGAAAGTACACCAATAGTTACTGCTGCTTTAG
CCCAATCGGGCATGCTTGACCATGCTGATTGTAATGCTTCAACCATCCAAGTAATCATTT
CAACAACTGTTATAATTATGGGAGTTATTGGGACAAGGATAGATTGTATTAATTTGCTTC
CAGTTACTGTTAATGCTGCCCAAGCATCATCTAATGTTACAATTTCTTGTGCGGACTGTT
CAAAACCCATATCGCTAAGTGTTTTGTTCATTGCTTGGAGTAATCCAGTTTTATTATTTA
TATCTCCATCCCATCCATTTTTCATGAGCATGTCTTGAGATATGCCTATTTCCTGCAGTC
TTCTGAATTGGCCATCCATTGCATCTGATACTGCAAGTATTGCATCTTCTTGTGTTCTTC
CTTCTTTTACGAAAGCTGAACTCATTACTGCAGTTGTTTTAGTAAGTTCATCCATTGAAG
CTTTAGGAAGTTTTAATTTCACTCCCATTTCTAATGCGGCTGCACCTACTGCATTCATAT
CTACTTTTCTGAAGCTATCTTGCATTTTGTCAACAGCCCCATGGAATTGTTGTAGTTCTC
CTTCAGTCATTCCTAATCTTTGACCGAATCTTTCAAAACTTGCAGCTGCATTTATTGATT
CTCTTGCTCCTTGAACCATACTGTTTACAAGGTCAAAACCAATCATTCCTACAGTCATAC
TTGCTGCTGTTCTAAGAAATCCAAGACCTCCACCTGCAGTTTTAGCTCCGTTTTCAAGTC
CTCCGAAACCAGTTCCTGTTTTTTGTGAACTTGTTCTTAAACCTTCTAAAGCTAATTGTG
CTTCTCTTGCATCTCTTGTAACATTATCTAAACCAGTAGTATGGAAGTTTAGCAATGAAT
TTACTTGTTGAAGTATACCAATTAAAACTAATAATATTGCTTTTAGTAGTTCTGCTGATG
CTGTTGTTCTACTGAATCCCGAACCATCAATTGAATCTAAGGCTGCATCTGTTGAATGTG
CACTTGTTCTAGCTCGATCTAATGCCATGTCTAATTGTGCTGCACTTGATGAAGTTGCTG
TTAATGTGTATCCAGTTATACTATTTATTCCACTGCTTAAACTAAGTGCACTGGATGAAG
CTTGTTTTAAACTTGAAGATAATAATCTGGTACTATTATTTGAACTATTCATCGAGGAAG
ATAATCTTGTAGCACTAGATGATGCTGTGAGTAAATTACCTGAATTAATTGTGTTGATTG
AAGTGTTAAGTCTATATGCACTATTCATCGATGTATCCATTGAAGTGGTTAATAGTTTTG
CAGAATTAGATAATTGATTAATTGTATTGGGATTGATTGAATTTAATGCTGTTTTTGTAG
AATTAGTGCGATTTGTAACTTCAGTTAATTCTTTATCTAACTGATTAGCAGAGTTTTTGG
CTTTATCCATACTTGAGGAATCAAATTGTCGCATTGCATTTTGAGCTTGTTGTGCTGCTT
TTTGAACTTGCTCCATTTGTGCTTGTATCTCTTTTAATTGAGCTTTAACACGGTTTTCCA
GTTCAATTATTAACTTTACAATATTATTGCTCATTATTCTATTCCTCTGCTAATTTCTTG
TTTTTTTCTTCTGACACGGTCTTTTAGAGAATGTGAAGCATGATTTGTACCTGAAGATTG
TTTATCCTGTTTATCACGAACTAAGGGTACTGCATGATTCAAGAATAATTTCTGCCTAAT
AGTTAAATCTGCTTGGTTTTCTGCTAACATATAACCGATGTCTTGAAGAGTTACTACTTC
AATAGCTATGTTTTCATTCTTCTTCACGAAATTCTGCTATATTATTATCCATTGATTTAA
GGTTATCTAAACCACTGAAAATTAATGCTTCATTTACAATTAAGTCTAATGTTCCTGCTT
TTAATTGTTCAAGGTCTTTTTTGGTGAATTTATCAGGATTGTCTGCATTGTCTAATACTT
TTAATGCAAGGTTCATTTTTGCATCATATTTCTTTTTTTCCATTGCTGCGAGACTGGTTT
TGAATTTTGTGGTGTCTTCTGCAAGGCTGGTTTGTTTTGCTTTGATGGTGGAAATCATGT
CGAAGTTTCCTACATCACGGTATGCTGATGTGAATTGGTTGTATTCAAGGTCGCTTATTT
CTCTTATTGCTACTTCATTATCGTACATTGGTAGTTCGATTATTTTTGTGTTTTTGATTC
CGCCGATGATCATTTCTTTTGTAAGCATAAATATCACATTTTTATATAATAATTTTTTTT
TGAATTTTAAATAAAGTAATTGAATGAGAATAATCATATTCTCATTTCATTTAATCTGTG
TAAACAGTGATTGTTTTTGTTTTACTTGCACCATCAATTAAAATATGTGCTGGTTCTACT
TTAGTTGCAGTTGGTTCACCAGTCAACTCTATATCATACACTCCATCTTCAGCAGTAAGT
GTTACTTCACCATTACTTGAAGATTCAGCATCAACAGTTGTGGATCCTTTCATTAATGAT
ACTGTGTAATCAGTTGTTAATTTAGCACCTTCCTTATCAACAAGTTTTATTGTTGCATCA
TAAGTTGGGTTGGGATCACTGTCTGCTTTTCTTGCAAATAATGCTCCAGCTAAATTTTTA
TAATGGTCCATTGTTGATTCAACTGTTTTTGATAAACAAACAATACTTAATTCATATTTA
ACTGGGTCTGCATTCATTGTTGCAGATGGTTGGTTGATAATACACATTGGACAATATAAT
TCGAATTTTTTATCACCATTTCTAATGTAGAACATTAATTCAAAGTATTCTCCTGCAAGG
TATCTTCTTGGTCCTTCCTCACTTCCCCAGTACATTTCCAAGTATTTTTCATCAGTATCA
TCTACTGTTAAGTTTAAACCAATTTCTCTTTTAGCTGCTCTTGGTATTTTACACATAAAC
CTTGAGCCCATTCCTCTTGCATCATCAGTATTAACATTGTTTTTGATTTCAAGACTTATT
TTATTGGTTTTACATCTCATTTCAGTCCATTCATCTTCACCAAATCTTCTCATTTTAACA
CTGTCTATATGGTAAAATGATAATGGTAATTTTCCAAAGTTAAACTCTGATAATTTTTTT
AATGGTTGTTTACTATTGATTTTTGATTTGATTTCTGCTGATGCAGTTAAGAATTCTGAT
TCTACTTCAATACTTAAACTGTCCATCACCATACCTAAAATTTCCATCTCGTAGGTTGCA
CATCCTGCCATCACAGTATAAGAAGGTAAGATACTACTATTTGTACCGTAGATAATATCT
CCTTCTCTTCTTCCCAATACTGCTTCAAGAAGGTATTCTAATGTTTCCATTTGTAAGTTG
CAACTGAAACTGTTTTCAGGTACATAGTAAGCAGCTACACCAGATTGATAATCCCTACTT
ACTACTGAATCATATTTTGTAAATTGCTCTGGTGGAGATACATCCATCTCTGATATTTCA
ATATCAATAGATTCTCCACTAAGGTCTTTTTTTGCAAATTCTCCTTCTTTTACTAATCTT
AAATATTTTAGTTCTTCTGCGGCTGTTACCATATTCTTTAGCCTCCATCACATTTTGAAT
TCTTAATTTTAAATCTTATTTGGAATGTTACACCTGCACTATACACATTTTTCTTTTTAC
CCAGACCATATGGAACCCATCCGAAATCCTTGGTTTTAATATTGAAAAATTCTAAGTCTC
CAAGTAATCTTGAACTTATTATTCTACGTTCTGCCTGTGAAACTATACTTGTAGCTTCAT
GAATACCTATCTTAGGATTTTTATTGTTAAGCACCTGTGAGGATAATATTATTTCTCCTT
CAAAGGTTTCACTTAAACCAGTATCAGTAATATTTTTAAATGGTTCATCTACCCATAATT
CCAGATAAGGTAATTCTGGTGTTCCCAGACTTACACCCATAAGAGATAAATGTTTAATAG
TGCCATCTTCAATCATTTCAGATAAGACCAGTTGGAAACCTTCCATTAGTTTATCATAAG
CTTCTATGAAATCTTTCATAATCCCACCTCTTTTAATGTTTCAGACACATATTTGTTAAT
TTTTGATTCAGTAGTTTTAACTGCATGAGGATAATAATGGTATCCCCTGAATGCTGCTAC
TTTTTTAGGTTTAGGGACAAAAACATCTTTACCAGATTTATCCACCCAATGCAGGGCTTT
TTTATTTTTAGCTGAGAAACTTCCACGACCATCATGAACATATTTTTCATAACCTGAAGG
TTTTCCTCTTGCTTTTATAACATACATTTCATCTGTTTCCCTTCTTGCAGTTATTGCAGT
TAAAAGATTGTAACGATTGTATTTTATCCTACTTTGAAGGTAAGTTTTAGCATCTTTACT
TGCTCCATAACTTACCTTTTTACCTAATCTTGGAGGTACCCGTAATATTTTTTCTTGCAG
TCTTTCCCATCCCGCATAATCAAAATCAAAATGTATCATAAAAAATCATCAACTCTTATA
AAAAATTAAAAAAAATGTTTAGATTGAGAAAACATGAATCTTAGATTTTTTAATGTAAGG
CTTCATTCTTTTTTCAATGTCTTCTGTGAAAATATTGTTTACTGCCTCATCAAAATCAAA
ATTTTCATGATCAGTAATTCCCAAGTCCTGTCTTACAGCATGGCTTCTAATAATGTTACT
GGTTAATTCAATAATGATTTGTATTACATCATCAGGAATTTTATCTGGTATATCATATTT
CTTTTTAACCCATGATTCCATTGCAGTGTAATAATCATTAATATAATTATCTAATTCAGT
AGTTTCTAAATTAAATAATTCTTCAGTATTTTTATTAGCTCCACTGTACTGTTTGATTTT
TTTTAAGGTGTTTTCATCCAACATAAATTATTCCTATGTTTTAGTCATTGTAATGGTTAA
TGTAGTTGAATCATCACCTACAGTATAATTACCTGTGCTTTCAAAGTTTTCATAACCAGT
ACATGTTGCACCATATGAATAAACTCCATAAGGAACATTACTTATTGTACAGCCTCCAGC
ACTACCAGTACCGTTACAAGTGTAAGTTTTACCATCAGTAATATTTGTTAAAGTTACAAT
AGCTCCTTCAACAGGATTTGTTCCATCATCAACACTTACACCAATATTTTTAGTACTAGT
TGTTGTGCTGGAGTTTATGCTTCCTTTATGTATGCAAGAGCAGATGCATCAGACCATGCA
AACTGAATATCAGCATACATTGTAGAAGCAATATAATACTTGTTAGATTTTAAATCAAAT
TCAGATTCAACAATAATATTATCAGGATCCGCCAACCATTGAATGTTTTCTTTATGAGTT
AAAATTACTGGTTTTTTAGTGAAACCATTACGAAGAGTACTGAAAGCTGGAATTGGTACT
AATGGTACTTCCTCAATAATGATGTCCCCATCTTTAGTAATAACTACATCATTAACAGCA
TACTTGTCATGATTATCTGCTACATATCTCCATACTGCTCTTTTAAAACTGTATGGTACA
AATGCAGCTACCCCACCATCATTTTTATATTTATCAGGGAATAAATCCAACATTCTACGG
AATTCTTTTAACGGATTGGAATCTGTTGCAGTTAAATCTATTGTTTCTTGATCTATATCA
GAATCATCTTCTAATTTTTTCAAGATACCATCATTTACTTTATATGAAGTTGCAACATTA
GATTCAGTACTTGCAGTATTCCCATAGATTAATGTTCTTTCTAATGCTCTACCATTTGCA
CTACCAAATTGACCAGTTAAAGTATTCATGAAATTCTTTTGCTCAATACTGTCATATAAT
ACAGTTCTGTGAACACCAGTTAAAGCACGATATTCTTCTGCATCAAAAGCCCTATTAGTG
AATGTTGGGTCCTGTTCATTGCTTAAAGTTTGTGGTGTACCACTGATTCTTCCAGCTTCC
AATTCAATATCAAAACTCATCATGTCCAATTCTCTTTTATGATTGTGAGTTGGAACAACT
TTAGTCTTGTTTAAAAATGCTGTTTCTTCTTGAACAGCTTGCATGTATTTATCTGCTTTT
TCCGCTTGAAGAACTCCTTTACCGAGTTTCCCACTTCCTTCTCCAATATCTACAAATTTA
AGGATAAAATCTTCATTATGCATGATTTTATCAGCTAACGCTTGTTCTACTTGTTTAATT
GTCATTTTATATCAAACCTTCTCATAAAAAATTTAATTATTAAGTATAATATCTTCTTTT
TTTCTACCAGGTCATTCCATTTGAATTCCTACCCATTCTCTCAAGGAATGATTTTTCAGA
AGCATTGCCTTTTGCAAGGTCAGGATCAATACTTTTACTAACAGTAGCATCTGGATTAAC
AGCACCATCAGCAGTAATAGTAGTATCCACTTTAACTTCTCCTCCATCATCATCTTTTGG
AGGTTCTGATTTTTTAACAGCTCCAGGAACAGGATCCTCTTTTGGAGGTTCTGGTGGTTT
TTCCCCACCTTCAAGTTTAGTTATCCTTGCATCCATAGTATCTAATCTTTTATCGATTTT
ACCTAGTGCTTCTAAAATTTTATTTTCATCTTCCACAGGTTCATTATTTTGTGCTGGAGG
TTCAGTTGGTTCTTGAGGTTCAGATTTAGCAACTAAACCATTAATTAATTTTTCAAGAAT
ACCTTCTGAAACCTGCACATTATTTTTTCCATTGTCTTCTTTTGTCATAACTTCCACCTC
TTCTGGTGTGATATATTTTTTCACAAATTCTTCATCATCTTCATATACTTCAAAAACCGC
CATAGGATGTGATGGTTGATCAACAATACTGATTGTTGATGGTTTCCAATCTTTAATGTC
TTTAAATTTCAAAGCCATATTTAACCTCCTAATCCTCTACTCATTTGAGCAACACTTTTA
TATGGTGCAGCCAGTATGCTGAAACCATTGTATTCCCCATCTCGGATTGCTTGTTGTATT
TCCTCATCAGTTACATCAACAGAAATAAACCAGGATCCTTTGGGGTAAGTGTTTCCCCTG
AAAGTGGTTGGTGATTCAAGAATATATGATTCTAAAATTCTTCCTACAGGTTGGAGTGTG
TGCTGTACATCAACTCCTAATCCCAGGCGATTGTATATTAATGATGCTTGTCTTATTGTT
TCTTCATCCAGTATGTCTCCTGTTGCATCAGGGATTCCTGGTATGCAGACTGCACCTTTC
ACAATCATAGTTTTTTTTGACTCCCCATTTTTTTATATTGGAAAAAAATTAGTTTTGTTG
GAGTTTTTTTTAGTAAAAAAGAAAGGAGGATTTTAAAAAAAGATAAATGATGTTAAGTTA
CTAAAATAAAAATATTTTTTTATCCCCCTTCAATATATAGGAGTGGAAAAATAGAAAAAA
ACGAAAAGTTAAATAAATTTATTCAACAATTTTGACCATAGGTTTAAATTGAGGATATTT
ATCAAGAAAATCTAAAGCATATTGTATATGATATTCCCTAGCTTCTTTGTGTTTCCCTTC
ATATTTTAATCTATGAGTTACAGCCCGTTCATTCTGATCCAAAGATTCAAACTCCGGATG
TTCAATACACTGAATTAATCTTAATCGATTAATATTAGGAATAACTTCACGTACAATACT
CATTAATCTATGAGGTAAACCTGAAGGTGTTGTAATGAAATCTCTTAATTGATCCTCAGA
CCAATTTTTAACAAATTCATAATTAGGAATCAATTTTTCAACCATGATAATGAACCCCAG
TATTTGTTAGTATATTATATATAAAACTTCCTATATAAAAATTACGATATCATTTCCCAA
TGAGCAATAAACTCTGATTTTTCACGTATTAATCTTAAATCAACATTTTTATCAAACAAA
ATTTCAAACTCCTGAGGAGTTTCAGTTATTCCTTCAATATACAGAATTTTAATTCCTTTT
GGAATGACAATATAATTAATATACTCCCCATATTCTTCAGGTTTAACATTTTCGGAAATA
GAAGTTGATAAAAATGAATCTGAATGATAATTATCTCCTTCTACAAATTTAGTCATTGGA
CTTTTTTGTCTTCTAACTACAACAAGACTTTCTTCTAATATTTTACCTTTCTTAAATACT
GATTTTGAAGATTTAATTGCTTTATTAAAAGAGATATAATATTTTGAATTAGCTAAATCC
TGCTCCCATAATTTAGAATATTTTCTTTTAATACGCCACCTCTTAACTGGGTTGAGCTCC
CCTTTTATTTCTCTGAAATAATTATTCAAACTATAATATCCTTCATCAGAATATGCTTTT
ATGAATTTATACTCTTCAGAAGTCAAATCTTCTTTTTTAATTCCATATTTATATTCAAAT
GCCAATTTATCTTTAGATGATTCACCAACACCATAAACTGTTTTATTACCAATTTTCACT
GCTTTATTTTTAGCTTTACTAAATTTAGTGATAGATTCATTTAATTTAAATTTACTGAAA
TCTCTTTTTGAATTTTGTTTTGGTTTTGAAGAAATATTAATCATTACTGTTTTTCTTTTA
ACATTTTCAGTGATTTTACTTCCTGTATTTTTAATCTTGTTACCTATATTCGATATGGTT
CTAATTATCTTATTCTTAATCTTAGAAGTTATCTTCAAAGAATTTTGATTACTGCTACTT
TTATTCTTACCATGTAAGTAAGAATTAGAAGGAATATTAAAAGAACTTTTACTTTTAAGA
TTACTTGGAGTACTATTAGTATAGCTTAACCAACATCTGCAATTAGCAACATTCTCAGCA
CCTCCAGCTAAATCCCCAGGATACATCATCTCAGTACGATAACTACCAGTAATAATAAAA
TAATCATCAATAGGAACACTCTCAATAAAGTTAGCACGATGCCAAATTCTATGTTGTCTA
GCACGACCATTCATCCAAACCTTATACTTATACCCCTTATCCAAAGCATGTAAAAAACTC
ATCCTAGACTGATTAGTATGCAAAGCATCACGAACAATATTCTTAGCATTCTTACGATAC
AACTCCCCATACTTCTTTTTAATCTCACTATCAGACAACTTACTTTCAGCTTCAATCTTA
GCTTTTAACTTATAAGCATCACTATCAAAACGTGAAGATAAAATCTTAGAATAATCCCCA
ACAGTTTGATCAACACTACCTTGAATAATCTTAGAACGCTTTTTCATTAAAGTATTAGCT
TGACCTTGCAAAGCAGTATCAATTATGTTCTCTGCAATCTTTTTACTATTCCCAATTTTA
TTTCCATCAATAATATTATTTAATAATTTATCTTTATAAGAATCAATACTTTTATCAGCT
TTATTCGTATGTTTAAACAAATGATTAAAACTTATATCTCTTTTAACCACATAAAGCTGA
TGCAATCTTTGAGTTAAAAGCAGATACTGAAGTTTCTTTTTAGAAATCATAATAAACCAT
TCTTATTAAAATCAGAATTCAAAGCATCAATCATACTATCCAATTTACCTTCAGCATCCA
TTGGAGCATCACCATACAAAACCTTATCCAAAGACTGATTATTCATAAACCTACAATTAT
AATACTCATCATCTTCACTCATTTTCAAATCAAAAGATTCACCAAAACGATTAACAAACT
CCCCTAAAGTCACTGCACCATTTTGCAGTAATTTAATTCCACGATCCAATACTTTATCTT
CCTCATCAAAATTAACAGGCAAATACTCAAGTTTATGTGTAAATATTCCAAATTCCTTTT
GAATAATAGTCTTATTAATCAAATTAGCCATCCTCTTTTGCAATGTAGCTACTGTAGATT
TACTATAATTTTTAAGCAAAGTTTCAGTACGATTACTAGCAATACCTGTAGACTCAGAAT
CACCTAAACGTTCACGAGGTACACGATGAATACGACGAATACGATCAGCCACACTACCTG
ACAGTTCTAAAAAACTGCCTTCTTTTTTCTCATCAGCAATTTTAGTAACATTAACACTAA
CATTATTTTCTTCACTGGGAATAGTGAAAACTAAAGCAGTTCCAGGCTCATTGGACACTT
CACGAAACTCATGTTCCAAATCCTCTTCAAAATCATCAAAAGTATAATCTTCTTTTTCCT
CAACATTACCTGTAACAGTAATAATATAATTCGGAATACCATGTGCTTTAAAATGCCCTT
GCTGATACTCAATAATAGCATTATCAGTTAAAATAGCATCCAACTCGGACAAATATTTCG
GTCTACCATAAACAAGACTTTCATTACTTTTAAGATTAAACCAGATTAAATCATTTGCAC
GATTTTCATCTGTAATATTATCATCCCACAAACCAGTTTCACGATTCAATTGCTTAATAT
TGTCAGGATCATATAATTTGAAATAATTTTCTTTATGCCCTATCTTTTGTACAACACGTT
TTTTATCACGACACATTCTCAGGTATAAACTGCTTACATGATTAACACTTTTCAACTCCC
CTTTTTCCCTAAGTAGTTCTAATCCTGCAAAACCAAAAGCTTCTAAATCTTCTAGGAATG
ATTCTATTTCCTCATCAAAATTAAAATCATTTAAAAAATCATCTAATCCTTCAGGTATTT
CTTCTCCTTCTTTAGAGATTATTTTTTTACCTGTGAAAATTGCATCTTCACATTTAACAG
TAATACAAATATCATGCAAACCACTAATATCACGCAACTTATCTAATTGGAACGGGTCAT
ACGCTGGGTCAATTACTTCTGTACCATATGTTAATTCATCTTTACTCATTTCTTGAGATT
TAATCTCATATTCATTTAAAACACTTTTAATCACTGAATCTCTTAAAAAGTTACTTTTAA
CTATTTTAACCATTATTAATTTCTCCTTCTACGATTTCTTTTTCTTAAAACTGTAGGTTT
TGTTTGAGGATATAAACCTTCATGTAACAATGATACACTATCTACACGGTCATCATGTGT
TGTTTCATCATCAGCTATTATTTTCTCTGATGGGAATTTCACAGCTTGTTTCATGAAATC
TTTCAACCATTTACCACGAACAAATAATATCCGTCCATTATTCATCCCCCGTATAGTTCT
ACTTGCTCTTATTAATTTTGATTTAGGAACACGAATAAAATTAGGATGATAGTCTTCGAA
TTCGTTTTCCCAGTATCTTTTAACTATTTTACCTGCAGCTGCAGGCTGATACTCAATCCA
ATTATCATACTCTGAATGTTCATCCATAATCCTTGTCATGTATTGTTCTAACTTACCTGG
CTTTTTTTGTGTGCTTTTCTGATTATGCACTACACCTACTTTTCCTTGTAATACTGTTGA
AAAACTACATGCTGTATAATCAGAAGTACTTTTTTCTGTTGCTGCAATATCCCATGTAAT
TACTTCTTGAAGAATATCCTTTTCAGTTAATAATTCATTAAACTCTTGTTTGCTAATTGT
TGCTGCTTCAATTGTATCATAATCAAATACATCTCCAGCTCTTATTTCATAATCCCAATT
ACCAATCTGATACTGATAATCTGCTTTTGATAATTCACGTAAATTAGCTAAGTATTTTTC
TTTGTCAATATATGGATTATGCCAGAAATTCATTTCAAAAAAAGGGTATTGTCCTTTAAC
AAATTTTTCATTTAAGTATGTTGATCCATCTGCATCAGCAGGATTACTAATATAGTAAAT
TGCTAATGGGAAAGTCATTAACTTATCAGTTCCTCTGAGACTACGATTCAGGAATTGCAG
GTTTACTTTTTCAAATTCTGAAGCTTCATCAACAATGATTTTATGATAAGCACGACTTTT
GAATTTCTCTTTGTCTTTTTCCAGAAGCATATATGAATAATAAATCCGTGCATCATTTTC
ATGGTTAATGAAACATCTTTTACTTTGATTGTGTTCTATGTAATCGAATGGTTCTGTCCA
ATGGTCCAGGTAATCTACTATTCCTCCAGTCGCTATTACATTATCATAAGTAGACCGTAG
AATTAAGCAGCGGTAGTATGGTACTTCATAATGTTGTAAGGCTAATACTGCTCCAAGCAT
ACTTTTACCAGAATATGCGGAACCTCCAATTAGTTTTCTTGTGTGGCGGTCTGCAATTGC
ATATAATTGTCGGTCGTATGGTGTTACTGGAATGTATGGGTTTTCAAGTATTGTTCTTCT
AATCAACTTTTTTTGTTGTTGATCTAAGTGTATTTTCTTGTAATCTACTCTCATAATTCA
TCAGTGAATTTTTCTAAATCATCGTTAATGGTTAGTAATTCTTCTAATTCTTTGTCGTTC
ATATGGATTACATTATCATTTTCTTTGGTGGATGTTTCAATACTTCCTTGTATTTGAGTA
TTATTATCTTTAATATCTGTTGGTTGACCATGAGCTAGTCTGAAATTTCTATAGATTATT
TCCGCAGATTTATTTAGATTTAGAAAACTATTTGCTCGTGTAGTTGGAGCATTCTCATTA
CTATTTTCTTTTAGTTCTTTTTGTATTTCTTTTAGTAATTCAAAATCATCATTGAAGAAT
TCTTGAAACTTAGCATTTCCTTGTTTGAATAATTCATAGTTCCACTCTCTTTCTTTTTGA
TCCATATGATTATCATATAATCTGCATCTTTCCACCCAATTCCATCTTGAAGAGAGTGTT
TTTAGTTGACTTAGTGAAGGTATGTTTTTTTCTTCAGTATGCTTCGATTTCTTCAATTTT
ATTATCTGTGGGAATGTTCTTTTGGAACCTAAATCTCTGTATTCTTTGAATAGTGAATAG
CTTTTACTTGGTTCTCCTTTTTGTCTTTCCCATGCTTCAGTCATAATCCCCCCATCCTTA
TATTTTTTTTGAACATTATTGTAATGTTTATTCTTTTTTGTTGAACACGAAATTTTAAAA
TACGGGATTTTTTATTTTCATAGTGAACAATATAATATTGTTTAGTTTTGAATTTTGAAC
AAAAAAAAGTTTTATTAGTTTTTTATAATAGTTTTAGGAAATTCACAATAACAAACTGAA
ATCCATCAATCAAAAATACAATCAAAGCACCAACAATTGCTATGAATACTCCACTTTTTA
TTGTGAATAATCGATTATCATCATTAGCTTGTTCTTCCTGAATAGCCAACTGTTTTGTTT
GAATTTCAATACTTTTATCCATTTTCTTTATCAGTTTTTCTAATTGCTTATTTTGAAACT
GATCACTTGCTTCCAGTTTACTTATACGAGATTCCTGTTTACAATATTTCTCATGTAAAT
CTTGAACTTGATTATGGGTCATTATTGTTTCCTCCATTGTTAGTGGAGGTGTAAGTTCTT
GAAATCCATCCAACTATCCCCCCTAATGCTACTGCTGCTAGTTCGTTGTTTCCAATATAG
GTGCTTAGAATTCCTATAATTATTATTCCAATTATTGCTAATGTTGTGTTGTTGAAGTTA
GTCATTATTATTTTCACTCCTAAAAAAAATTTGTTTTTAAAAAAAACATTGGGGAAGGGA
TTTGAACCCCTGCGATACTAGTGTATCATTAGATTAGCAGTCTAACGCCCTACCAGGCTA
GGCTACCCCAACATTGTTGGTGGAGGGAATTGCACCCTCAACTTTTGAGTGGTTACTAAG
TTACAATAAAAAATAAGCTATTACCAACATCATGAACATGTATTAAAGGAGATAAATATT
TATATTATATTTATGGGAAGAATATTTTTTTTTAAGATATGTGATATTAATTATTTAATC
TTTGGAGGATACCTATATATTTTAATGAAGAAAAAAATCCAATCATTCTATTATTCATTA
AAAATTTGCTACCATTCCCATAATAATTTTTAAGAATAATAATAAATGAAAAGCGAATAA
TAAACGGATAAGAATATTATTTTTAAATTGTCAAATAATGATAATGCGAAGCTAATTGTA
CAATAACATATGATAAAATATTTTTTATAAGTGTTTTATTACAAGACCACATTTTTTACA
AACATACTCATCATGCCACTCATCATAGTAAACCTTTTTGCAATCTTTTTTCAGTTTTTC
ACAAACAGGACACTCTATTTCTGCATGTTTCATTTTTTAAGGGAAATACACACCCGCAGT
TTGTGCAATTATATTCTGCACGAGATTCATCCAAAACAGTTAAAGGGGATTCAGGAAACT
TAATATAACAAGATGTCTTATGATTTCTATAATGTTTTTCTTGTTTTTTCCTTTGTTTCC
ACTTCCAGTTACAAACAGGACACTCTATTTCTGCATGTTTCATGTTTTTAAAAAATGTAT
TAGCATCCAAATAATATTATCCTCCCATATATAATTATAATAATGTCTTTTATTTTTTTT
TCATAAAAAAAGATAAAATTATAAAATATTTTATCCCCTTCAATATATAGGAGTGGAAAA
ATAGAAAAAAAGATTAAAAATAAAAAAAATTTAATCACTTAATAAATAACTATCAAGAAA
ATGCTCACCACGACTGATATGTCCCTTACTTACAATCCATTTAATCATGGGATCCATCAA
ACCTTTTTCATATTCTTTAATAAAATAATTCAATGTCCAAAAAGTAACCTTTTCATTAAA
AATTATCCTTTCAAAAAGTTCTTTAGATGTCTTTTTAAATTTTTTTCTATGAAAAATAAT
ACTGTCAATTTCATCAATAGTTAAATACAATGCAATATCTTCACCAGAAATATAAATACA
TTCCTTATCAACATCATACCAATAATACTTCTCATTAGGATTTCTTAATAATTTAAGATA
CTTGATAAAATTCATATTTTTACTCCTATTCTTTTTAATTCTCTTTTCACAGTTCTTTCC
TCCAAACCAAAATCACTCAAAGCATGCTGTGAAAGAAATGTTGTACCAGGCTCCCTTAAC
TCATTACTAATTAACTCCCCATCATTAATTAATTTCCTACGTTTTTGTTGATACTTTGCT
TTCTGCTCACGTACCGCCCAGGTCCTGCAAGCTTCCCTGCAATAACCTGTTTTATTCTCA
AACTTAATAAATACTCTGCCACAGTATTTGCATTTGCTGATGTAGAATCTTGTTTGTGGA
TCATTTAATGTTTTCTTCAATAACATCCTATCATCTTTCTAAATTTTTAGATAGTAAGTT
TTTCTTCACTTCAAAAACAAACATGTGAAAAGGTAAAAGTTTACCCTGATATTTCACATA
TTCATTAATTACTTGTGATTTTTTTAATGCTTCAGTTATAGTTTCTTGTTTTAATTCTTC
ATTTATGCATTCAAAATTGGAGACTGTTCTACTTAAACTAATGTATACTCTTTCATATAG
ATTAAGGTTATTGTTTTTCACATTCTCCACCACAACAAGAAACTGAATTTTCAGAAGTGT
TGAATTTACCATCTTCAATCATTTCAATCAACTCATTAAGAATACGAGTAACATGCATCA
AAGTATGAAGATTAATACTATGAATTATAACATCATCATACTCCATGTCTCTCAAACTAC
CTGTGCATTCTTTATGAATAGTAAAAGATTGTTCTTCAAATTCCCTGCTTCTTGTTTTAA
TAAAATATAGTAATAATTTCTCATCAATCATACATATTTATCCTCCATGTATTTTTTAAC
TTCTTTCATAAAAACTCCTGATTACAATATAACCTATCTTAATCATCAAAAAAATCAGTA
AGACATAACACTAAACAACAACTAATAATTACACTTACACTAAAAACTACCCAACCATCC
ATCATACTATATTCTCCCCCCATTATCTCCAACTTGTTATTAACTCATACTCCTCATGAG
TTAACTTCTGTTTTAACCTTTCATCAAAACTTCTTACATCTCCAGGATTATTCTCATCCC
AACAAGACTCAATCTTGGCAGTTAATTCATTAATTTCTTCCTTATTCATTTTCAATATCC
TCCTATTTAATTTTGAAAGGTTGTAATGGTAGTGTTGGTCTTTCAAATATAATCTGAAAC
AAAACAGTATCCCCACATATATCTGGATAAATACTTTTTAACTCATGTTTCAACAAATCA
ACATGCCTATAACCTTCAAACCAAGCATGATTATGTTTCAAATCTTTAAATTTAACCTGT
TCAACTCTCCGTACTACCCGAAGCAGACAAAAACCCGTCCCCTCAAACCAACATTCTACA
AGGTCACCTTTTTTCAATCCTTTATCAGATTTTCTTATTGTAGACATTTTTTTCCTACTT
CTTAAAGGTTCAAAGAAATACGGTTTAAACTTCAATGTTTTCATTTTTATCAACTTCCTT
TTTTACATATCTTTGCTTCTTTTTTATCATCATCCAACACTATATTATGTTCTTTTAGGT
ATGATTTTCCAAATAATTCTTCAGCATGTTTACATAACAGATAAGAATCAATTACTCCTT
CAGCAGATATTATTCCTTCTTTTTCTAATTTTTCAAGTATATCATTACAACTCATTTTTG
GATCTCCTTTTTATTCCGGACTGGTTGTCTTTGACTTGGTTCAGGAACACCACACCTCAT
ACGTACCTTTTCAGCAAACTTAACAAACTCATCTGCAAATAAACTTATTCTAAACCACAT
CAACTGCCACCTTAATATAATATGTTCCTCATCTTCTTTCTTCATGATTCCACCTTTTTT
AACCTAGTTTGGAAGTTATTTCCCCCATAACGCCTAACATGATCTTCACAAATGATTCTT
TTCACAAAGGAATCTTTTTGATGTCGAGTTAATCCATACCTTTTCTGCAACTCATCAACA
GAGATATTTAATTCATGTTTATAATCTCTCCTGAATTTCAAATATGTTTCTTCACCTTTA
GAAACATCATAATCCGTTTCAACAATATTAAAAACTGGTTTATTCATTTTTTATCTCTCC
TTAAAAAAATGTAAACATAAATGTTTACAATTTAAAAAATATTTATAGTGAATTACGTAC
GTATACGTACGTACGTATGTGCACGTACGTGGTTTCCTACAGGTTCTTCGGAAAAATGTA
AACATAAATGTTTACAAATCATTTTATCACCAAACCATAATTCCGAACATCATAACCCAA
ATCATACACATGATCCAATAATGACTTATACGGAACTTCTTGCTGCTTACTAATATTCTT
CAGCTGATCTTTACCAATAAAACCAGCATTATCAACAATCCTATTAACTGTACCCATCAC
CCCATCAAAGACATTTACACTTCTTTCATGTTCTAAACGCTCAGCACGAATCTTACATAA
TCTTGATTCACGAGCATTAATCTCATTTTGCAACTCAGCTATCTCTTTTCTCAAACTATT
TTCCTCATCTTCAGATAAATCAATAGCTAATCTTAACTGCTCTTCTAAAAACTCACTTCT
GGTTGTCCCCAGTTTGTTTTTAGCTTCCCTCCATACTTCAGAATCAACCATGAATGAAAC
CTGTATTTTTTCACTCATACATCATCACCTAACTCTTTCATGACAGTTTCTAATTGCATT
TCCTTTGCAATTAAATCTAATTTTAAAGACCTGATTTCCTCTTTTAACTGGTCCTTTTCA
ATTAACCTTTGATTTTGTTTACTCACATACATATCAATGCAATGTTCCACGCAGAAACGT
ACAGTTAAACCATACTTTTCCATTAACTTTTTTGCCCTTGTAGTAATCCTACCAGATACC
ACTTCAGGGAAATTATCCTGAGTATTCATACTCTTACACCTAACATTTTTTCTGCTTTTT
TAATATGTTTGCATTTATAATGTTCTACTTTTTGATGTCGGTAGAAATAATCTTCACAAC
TACACCACCATCCATCTATACTGTCCCAGTTCACAGTATTGGATCCAGTATTTCCTGATG
CCTTGAATTGTATGAATATTATTTCTACCTTAGGTTCCATAATTAAACAGCAACCTCTTT
TTCTTGATTTTTAAACCAGTTATATGCTTCTTTGTTTTCAGCTTTGGTCAGTTCACCATC
TTTGAATAACTTCATTCTATGAGTATTAATCATAGATGGAGTTATTTCCAGTGAACTGTC
CTTTTTATGAATATACTCGCGGATTTTCTGCACAGCACCAGTAGCCTCAACTTTCTCAGT
TCTTGCAGAACTACCCTTAGCAGTTTTAACATTAATTTTATCAGAATCCACAACATCTTT
TTCAACAATTAGAAACATGTTTGCTAATAAATATCTTTTTAAGTAAGTAATGTAACTGCC
TTCACTTTGCATTACATTCATACCTTTATTTAAAGCAACTATTTCAGGCATAGGAACACT
GGTGATGACAGATTCCCCAGGTTCATTCCAATTTCTGATTTTCAATTGAGCCAATTCATT
TGTAAATGAAAACTCAATGAATAACTCCTGTTCATAACATTCACAAAAGATTGCAGGCAA
AATATCTTCTAATTCATAGTACTTAAATTTCCCAAATTTATTGTACCCACTTTTAGGTAA
TTTTTTATGCAGGAGATTCTTTTGAATTCCTGCAATTTTCTGATATATAGTCATAAAAAA
ATAATCCCCCTTTTTACATATTCATCGACTTTTTCATCAAATCCCTAATTTCTTTTAACT
GTTCATTTTCACTTATTTTTCTAAACATGGTGTTCAGAATATATTCATCGTAATCTATAT
TTTTTTCAGCACATAACTCTTCCATCAGTGCTTTAACTATTCTAACTTGATTTTTCTGTA
CAATATGTTTAAAGATCATATCAAAAAATGAATTCAATTCTGCAGGATTTTTAGCATCTA
CATTATGGTCTAAAAAGTGGATTGCATTAGTTAATTTCTCCTTTAACTCTTTTTTGTTTA
TGATTTCTTCAGTAAATAGTTCTTTCTCCATTAAAATTCCTCCTCAATAATCTCCAATAC
TTTGTTTTCATCAATATTTTCCAAAGATGCTTGAGAGTGTGGAATGAAACTTTTTGACAC
CCAGTAGTCATGATACTGGATTTCACCACCACACAGTTCCTGTAAAGTATTCATAATATC
ACGTGTGTTCTCATAAGTACTGTCATGAAAAACAATTACAGATAAGAAAATACCATGTAA
GTTTCCATGAGCAGAAATACGTCCGCCCATTTTTTCAACAATTCCTAATAATTGATTGTA
AAACCTGTTTTGCTGATCATAAGTAATCATACTAAACACCCATTATTATAAGGATCCATA
GGCCCTACTACAAGGAATGCAAAAATGAATAATAAAATTATTAAAATTATTCCTTTTGCA
AAGATTATTTCAACTTCATACTTTTCTCTCCAGGACACTTTTTTATGAAGTCTAACTGGA
GTAGGTTTATTGTTGAAGAGACTCATAATTGAGCCTCCAACACTTTATACTTAACTTCTT
TTTTCATACCAGGAGTATTAAGGAAATCTTTCAATTCCCTTAATCTGGTTTGTTCTTCAA
CATACTCTGCATACTCCCCGTATAATGTTTCGAACAATTCTCTTTGTTCAGGATATTCCT
GTGCTAGAAATACGAACAGTTCATCTATTTCACAGTAATCTTTGTACTGTGACTGGTTTA
TACGATATAACGCATTACCAATCTTGTTTTCATTTTTCCAAAATTCAGGAGTGAACATTC
ATTTACCCCCGTAAGCTAGTTCACCTGCACGAGCAAACTCCAACATTTGATCATCAGACA
ATTCATAGATTTGAAATTGCTTATATTCATCAGCCAACCTATGATCTATTTCTTCCTGTG
TTTCATAATCCTCAGACAATAATATCTGTTCAAGATAATCAAACTCGACCTCATCTTCAG
GTGGAGTAATCGTATACATTGAGTTGTTGTATCTGCAACTCATACGCCCACACTCCTGAA
TTTTTGGAGTTCTTCTTCATAGACTCTGTTTTGAGCCTGAAGCCTTTTTGCTTTTAAAAA
TACATGTTCAATTTCTTCTTTGGAAGTTAACCTTCCAAAAACTTCAATCTTATTATTATC
CATATTACCAACTCGTGGATTTTTTAGAAGATCTTCTTCAGAGTTGCCGCTCTGATTTTT
TCTTCTTCAAATAACTATTATACTTAATACTATATAAAGGTTATTATATTTACCTTTATT
AAAGGTAAATATATATACTGCAAAACAGAATATATTATATAAGAAATATAAAAATGGAGG
TATGCCTATTTTAGAATATACAACAAAATTAAGTAAAGGCGGACCTAATTCTATTAGGTC
AATAGTCCCCCAAGATGTGATAAAATTATTAGAATTAGAACTGGGGGATTCATTACATTG
GATTGTAAATATTGATGAAGGAATAACTGTATCTATTGAAAAAGCTGAAAAATAATCAGT
TAATCCTTCACAAATATTTTTAGTAAATATTAAATATCTTTTTTTATAATAAATAGTATT
GGTTACTAAAATTTTTATCAACTCGTGGTATCTATTTTTTGTAGCCTCTAGTTTTTGAGA
GGCGAGATGTTGCCGCATCACAGACCCCCTCATCAACTAGGAATTACATTTACTTATTTT
GCATTTAATTGTTCTTTTTCAACCTTTTTTTGTTCTGATAACATCATAAAATATGTAAAG
TATAATCTATTTACAAAACCATATTCCCCACTATTTTCTCTCCCAATAGACTCTATAATC
CCAAGGTCTTTAGCTCTCTTTAAAAAATCATTAAAAGCTCTCTTCTCCCCATTTGTCAAA
ATATTTTTCACATCTGATTTTTTAAAATCCATTAATCTATTTTCCCCTAATTTAAGAAAA
ATATCAATATATGTATCGCTTCTAATTTTATTTAATTTGGATTTTAATTGTTTTTTAGCT
AATTCATCTGTTGCATCAGTAATACCAGAAAATACTATAGATTCATTTATTTTTAAATTA
TCTTGAGCATTCCAAAAAATTGAATCACCAATTTGTTGCATTATTAAAGGCATACCCCAT
GAAAAATAAACCATAGGTTCTATAGAATTATATCCATCTTCAAACTCAATGCCTACATTA
TTAAATGAAGTTGTGAAGAAATCTTCAATATCATTATCCTCTAAATTATCAATTTCAATT
AAATTAAACATTCTAGAAAATGATTCATTAATTGAACATAATTTTTCAAATTCATGAGGG
TAACTTATTAATGTGAAAACAACAGGCATATGATATTCATTAACCAATAATGTTTCAGAT
AATCCTTTATACCAATCTGTAAACTCTTCATTATCAGATAAACCATTAAGATCATCAACA
ACAATAAATATTCCATAATCTTCAGGTAATTCTTTACAAGTAGTAATCAAAAAATCTGCA
AAATGTTTTTTAATATTATGAATTAAATTAGTATCATTATCTTTTAAAGATACTCCAGTC
CCTGCAACTTTAAATTCATTAATATTATTCAAAATTTTATCAATTACACTTTTACCTAAA
TAATCCTTTTTAAATTCTTTTAATAATCCATCAAGTAACTTTTGTATTAATTCATCAATA
GTTTTTCCACCACCATTGTTAATATGGATTGGAATCATTTGAAAATCATCTTCTGCTTTT
CTTGAAACATAATTTATAAATGATGTTTTACCCATTCCTCTCTTTCCAGTTATGAAAAAA
TGTTCGGGAGCTCCTTGATTTTTAACTTTAGGTAAATATTTTATAATTTTGTTGATATCT
TCTTTTCTTCCTTTAAAATTATCTGGAGATACTGGTCTCCCTGGTTCAAAAGGACTTTCT
GGGAAATCTTGTGACATGATATTTTTACCCCTCAATACTCATTTATTACAATTTACATTT
TGTATTTAACTGTCTTATCAAATTTTTATTACTGTATCAGCTAGAAAATCTGGTGTAACA
TTCCGTTTCTTTTTTTGATTTAAAATAGTATTGGACCATTTACTGAACTGTGTAGCTTCT
TTACTATTGACGCGATAGCCTACGGAAATCATAGCATCTAAATTATAGAGTATGGGTTGA
GTATTTGCTTCAGGATTAATTTTAATAACGATTCCACTAATAGTGGAATCGTTTGGATTA
ACAGTTACTTCATCTTTAACAAGTTCTCCACTATCAAAGATATTATTTAAATGTTTACTA
ATGTTTTTTGGTGTTTTATTAAATAAATTAGCCATTGTTTTTTGTGTTGCCCATAATGTT
TCGTTTTCTTGATCAATGATAACTTTAATTGATACTGGCCCATCTTCACTTTTATATAAA
AATGTTTCAATAAGATTACTTTCCACCATGATTTATATAACCTCTACAATTTTACTCTTC
AAAAAAGTTTTTTTATTCTTGTTTTTATATTTATGTTATAAGTTAATATAAATGTTTCTA
TTCAAAAAATAAACCTTAAAAATACAGTATATTCAATGAAAATAATGAAACACTAAACAT
TATTCAGTATGAAAAAATGAAACGCATTGTGTTCTAAATTTTGGTGTTTAATTAGTTTTG
TGAACTGTTAGGTTATTGTTCATTTTTATAATGAATATTTTTTATCTTTTTTTGTTATTA
AAATGTATTGATATTTTTAATTAAAAAAATAGAAACCTTTATATACTATGTAGTACAATA
TAATAATAGAAATACATGGAGGTGAAAAGTTGTCAGAATCAGAGGCAATAAAAATCATAA
TATTGTCTTTGAGTATAATCCTAGAAATACTCAAAAGACAAAAACACAAGTAGGAGGTTA
ACCCCCTCTTACAATTATTATATTTTTTATTGCTTCTTATATAAATTTTTCTTAAAAAGG
AGGTGAAAAACAAAATGACAATAACAAGCATCATAATACTAATATTATTAATCATATTAG
CAATATTAATCTACATTAATAGAAACCAAAAATATACTTGGATAAGTTTAATAATATGGT
TGGTTATTTTAATAAGTTTCATCTACGAAACATTAATCTAACCCCCCCATATTTTTTTTA
TATTATAAGCCAAAAAAAAGGAAGATGATAAAAAATGACTGACTACACCAGCATAAGAAT
AAAAAAAGAAATAGCTGAAAAAATACAATTAATAAAAATACAAAACAACTGCAAATCACT
AAACGAAACACTAGAACAACTAATACCCCGAACAGTAAACGAAAACTACGAGTTCATAAA
AGAACAACCAATATTCACAATAAACAACAAACCCATAACATTCACAGACCTGAAAAACAA
CAATACAGGCAAAACATGGGGAAATGAAAAACAAAACGCAACAATAGTATTTAAAGATAA
ACAAGGAGCATTTATAAGATTCAATGATGAAGATGAAGTATTCTTAGAATACTACCACTT
CATCTAAAACCATTCTTTTTTTAATATTAAAAATAAATCCATAAAACCTCAAATTTTTAA
AGTTTTAAAAATTTGTTCCATAAGGTACATAGCATCATCCAATTCACCTTCAGAGATATT
ATAAAAAGGATCATAATCTGCTTTTTTTCTTAAATTAAATAAATCATATAGTTTATTCCT
AACAGAATACTGATTGACCTCAAGAAGTTCATATTGAACTTGCCCATGTTCTGTAATAAA
CTCATTTTCACCAAAGTCTTCTTTAGACAATGGTTTAAATTTATAAACTTCCTGAAGCCA
CAAACTAACATATAAATAAGCACTGTAATAGCACCTATTGATAATAGTACTAAACACACA
ACGATTATCGCAATTTTCAGGGATTAATTCTTCTTTATTTTCATATAACTGCTTAGTGAA
TTTATATAATTCATATTGAGGGTATTTTTCAATATTAATGTATAAAACCCCCAATAAATT
ATTTAATTAAAATATAGGTATCTTTATAGAAATCAAACATATTATTTTCCTTTGAAAAGA
TAACCATATCATTAATAATTTGGTCTAACAGAGAATATTTTTCTTTCCTATCAATATGAA
ATGGAACATTAATTGTAAATACTTTTTCAGGATATGATCCATCATACACTGTATCAATAG
ATATATTTTTTCCTAATCCTAAATTGTCAGAAATTTTTATAAGTTCATCATAAAATTCAG
TGATTTTACTTTCAAAAGACTCTGTGGATGATTTTTTAAAAAACTTATTTTTAAAAAAAT
CCCCTACAATATTAGAAGGTTTATTATATCCATAATTTTTATCAACTGAATACACCATAG
ACACCACCTATAAATGTTTATTCTAAAGTTTTTCTAATAATATCCAATGTTTCAGGATTA
TTTTGAACATCAATAAGAACTGTATTACTTTTTATTTCTTCATCATTCCTATAAGATAAA
GTAAGAATATCCTTATTTATTTCCCCATCTTTTAAAACAAAAGCTGCATTTATAGGTATA
ACTTCAAGATTTTCAAAGTTTTCTTCAAGTTTTTTTAAAAAATCAACATTAAAAACTTCT
TTAAGGGAAATCCCCTCTTGAAGCACTCTTAAGCTTCTTGCATCCTTTTTCATTTAACTT
CCTCCAAAATAGTTATTTAATAAATATCTTTTATTAAACATATAAACCTTGTCAAAAAAT
TTTTAAATTAATAAATCTTCAAAGTCATTTTCTTTGAAATTTTCTTCTTTTTTCTCAATA
TTTCGTAATCTTTCATCAATATCATAGATTAATTTTTCATAATCTTTGATTTTTTCATCT
TTTTCAACAATTGTATTTTCTAATTTTAAAAATTCTGGAGATTTAACAGTGATCTTTTCA
ACTTCTTTACTAATTGATAATGCTGGTAAATGTTGAATATATTCCTGTTTTAAATCAGCA
GGATTGGTCATGAAATATACTTCATCAGTACTGTTTTTTGATTTGCCTTGCAGGTCATTT
ACTTTGTCCAGACTCATACCATCATTATATAGTGTTGAGGCGTGGAATTTTCTGAGCATG
TGGCTGCGGAATCTGTTGTAGTTTCCTGCTTTTCCTAATCCTAATTCATTGTTTATTTTA
ATGAATTGTTGGTTTAGGTAGTCTTCATGTATTTTGAATAGTTGGGATTCTGGTGTTAAA
TTTTGTCTTGATAGTAAGTGATGATTTATAGCTGTGACTGCTTCTGGACTGCAATAGGTA
ATATAATATTTCTGTGTTTTTTGCCTTAAAATATTAAAAGTAGGGACAACATTATCAATA
TTATTTAACACATCTATCATTTCCATAATATTATCTGTATTATGGTATTCTTTAGTTGCG
TTCATATAGTCCATTACAGTTAAATTTAATGTTTCTCTTCTGGCACAACCTGAACTACTC
ATGAATAATATAATAGCTTTCATGGTAGAAGTACATATATTAACTGCTTCACGAATAATT
TCTTTATCAGGTAAATCTTTAAAACTGATCGGTTTGGGATTATTATAACTTTTTTTATCA
ATTCGTGGCAGATCATGTATTTCAATTTCAAAATATTTATATACAACAAGAACTGGTGTG
AAAGTATTTGATACAGTGTTATAATAATAATTATCCATCAAATACTTGCGAAAATTTAAT
AATCTTCTTTTTAATGTACGATGTTTCCATCGTATACCCTGTTCTTCTTCTTTTTCTGCT
TCTTCGATTAATTCGGCTAATGATAACTTATTCAATTCACAATATTTATTTATTGAATGT
TTGTAAATATGCTGTGTTGTTTTTTTATGATTTTTAACAGAATGGATCTCATTTAGGATT
TCTTCATTGGTTCTCATATTTATCAAATCTATTTTTCTCTTTTTATATTGTTTGATGTAG
AGTGCATTTGATAAGTATGTGATAATATGTCTACTGTCGTGTCTACAACGAGTTAATTAT
AACATACTTAAAACATTCTAATCTATTAAAATATTATATCAAAAATAGGTTTTAAATTTT
ATCCCCCAAAATTAAACCCTTATCCTCGTACTGCGTTTATAAAAAATGGAAGTAATTAAG
ATAAATTTCTCCGTATTCATCTTCAAATCTGATTAATGCTCCAAGGTTATCTTTATAGAT
TAATGTTGCTTTTTCCCCATTACTCCATTGTGTGCCTACTTCCGATTGTTTTAATTCATT
CCATGATACATTTAAAACTGTTTTTTTGTTTATTAGTGTGAATGCTGGTTGTTCTTGTAT
GTATTCTTCAGGTGTTGATGTTCCATCAGGTAATAAATGTTTTACTGTTTCATTAACTGA
TTTGTATCCGTTGATTTCCTGTATTTCTTTTATCTTGTTAATTAAATTTACTTCAAGTTT
TATAGTACTTCTTTCCATTATTTAATTCCTCCTTTAAAAAAATAATAAAAAAATTATAGT
CTTTATATTAAATGGATTATAATGATGACTATAATATTTATTATAAGCATTATTGCGTTT
CGTGTCCTGTTTTCTTCATTTTTCATTGAATATAATAATACTACTGCAAGTATTATTAAA
ATTATTTGAATTAATGTTAATACTGTGGTTATCATAATTCCCCATCCCTTTTATTTAAAT
ATTAGTAATGACATATAATTTATTATAAATTATTTTAAAGAGGGATTAATTTTAATCCCC
CATTAGGGATAATATTATTGATATTATCCCCATAACCATATTTATTAGAAAATATATGTC
ATTTTTATCAATGTTTTTCACCTCCTTGTAGTTCTATTATTATATTGGTTACTATACTAT
ATAAAGGTTTCTATTTTTTACTACTTATTTTTATAAAAAAATAAAAGAATTGTTGATAAA
ACAACAATCTTATTGATACAAATCTGAGAAAATCCAAGCTGGATCATACGCTAAAATCGT
ACCATTCATACACCAATTACTTGAAACACTGTTTCCATCTAAAACTGCTGCAGGGTCACG
ATAAATCCAAGAACCCCCAGTATACTTAGAATGTTTCAATCTGAGCCTTACATGCCCAGT
CCCACTTCTACATCGTACATGAATGAATTGTACATTATAACCTAATGCAAGTCCTAAACG
GTAAAATAATTGACTTGAATCAGTACAATTAACTCCTTGCTTATTATATATCTTATTAAT
AGTTTCCTGAGTATTATATTTGGAGTTATAATAATATCCATATCCTCTTCCTCGGATTTT
GTTCAAGCAAGAGTCTATATCTGTTACACCACCAAATTTGTCGGTGAACCTTTTTAATGT
GATGTCTGTGGTGTCGCTGGTTGTTCCGTTGCCATGTGGGTCTTCAAGATAAACAATAGC
TGGACTAACACCATTTAATACTTCATAAGCTGATACACGATTAGCCATACTAACATAACA
ATTCTTATAATACTTTGTTCCATCAGTATCAGACACATAATTTGGGAGGAAACTTAAATC
TTTATTATCAAATATTGCACATTCAACAAACTCTGCAATAAGACTGTCTTGTTGATAATT
CGTACCTCTCTCTTTAATTGCTTTTATAATTGTGCCATAATCAGCTACACGATTCATGTA
ACTATACACTTGTCTTGTATTAAACTTTTTATTATTATTCACTAACCAATAAGCAATTCT
ATTCATCAAATAAGTGTATTTTGTAAAACTAATATGTTCATTAACCATTTATTTTCATCT
CCTTTTTAAATTTTAAATCATCTAAATATTCCTTTAAAATACTGCATTTTATTATTGCAT
CTTCAAAACAACAAGGAACCTCTTTATCACAAGGATCATAAAATGCACATTTTTCACATT
CTTCACTAATCATTCCAAAATAATCCTCCTATCAATTTTTAATGTACAATCAGTAATCTT
GTTTGTTCTAAACGAGGATTCTCTCCAGCAGCTCCTCTTTTTACAATACCTTCCAACATA
TAACTTCCAGGGTCTAATCTAATCGGCAGTATTGCTCTACCATTAGCATCAGTTGTTCTT
TCATAAGAAACACCATTTACAATAAAATTAATTACCACATTTTCCCCTGTGTTAATTTCA
GGATTGGAAGAAATAATTTTACCGTAAACAGAATTTCCATAATTTAATTCTAGTTTAACT
CCATCTTCTCCAGCAAGGTCTGATAAGCGACCCATTACTAATCTAGTGTTTTTTAAATCA
TCCATTGTGGTTTCTAATATTTCTTGTCTTGCAAATGTTTCAGAATGTTCATCATCCACC
AAATCTGCATTTAAACCTGAACCTGCTCCATCCACTTCTTTTAATTTATTTAAAATAGTG
GTGGGTGTTTCAATGTTTCCATCTTTACCATCTTTTCCTGCAGGTCCAGTATCACCTTTA
CTTCCTTTATCACCTTTAGGACCTGTTGGTCCTGTTGGTCCAGTATTGCCTTTAGGGCCT
TTGAAATTCCCCAGTAATATTTTTTTAACCATTAAAAAAAACCTCTTCATTTATTTAATT
ATACTCCTATTTTCATTTTCACACCAGACACATTATAACTCATACTTGTACCGCTACTCC
ATGCACCTGCTTTTGTAATTAATCTCCACCTATTAGGATTAATTTCTGCTCCTCGACTCT
TATTGGCTGTGTTAATTGTTTGAGAATCATCACATAATACATAACTTAACTTATCATGTC
TTACTTTGAAAATTAAACCAAAATATTTTCCTGCAACATTCCCATATGTGCCCTTTTGGT
CGTAAGTTCTATTTGTTCCACCATCACAAACTATATCAACCCATGTACGGCCTTGCTGAG
GGGTGCATACTACACTTATTTTTTCGTTTGTTTCTGGGTTCAATAAGATAAATTGTATAT
TAGAGTTAGTTCCTAAGCTGTTAGGACTCCATGATAAAAATAAGTCAAAGTTTTCAAGAT
TAAAAGTATCTGTACGATACCCTTCTTGAGTGCTCTGCTTGTAGGGTATTTGTATGGTAT
CGTGAACATGTGAGTGAGTAATACTTTTATCTGCAATACTGCTTCCTTCAGGCAATATAA
CATCAGATAAAAAGAAACTAAAATCTCTTGTATTAATATTGGTTTTACCATCCAAGCTTG
ATTGAAGATCAGTAATATTGCTGATACTGTGACTGTGACTATTGTTAGCTTTACCGTTCA
ACGCATTAGTCACGATTTTATTCTGTACAGGATTAATAGAAGAACTACTTAATGATGAAT
CAACAACTGTTTTATTAGCACCATCATCAATATTTTCCAATTTTTTGTCATGATCATAAA
CAAAACCATTTACACTTGACTGTCTATTAATTCCATATTCATACACCCACATGCCATCAT
CACTATCCTGATCAAGGTCTATACTATCACTTGTATGGCTGTGGTTAACATTTGCTTTAT
TTGCTAATTGAGTATTCATTTCTGTTTGTGTAACATACCCTGATAAGTCAACTGTTGTTT
CAGTTGAACCAATTTTTTCAAATTTATTATTAACCCAAATATACTCATCATAAATATTAC
CCTCAACAGAAGCTGAAGTATTTAATTTTAAATACATTACTCCAACTTCCCCTGTAGATG
GTAATGAATTTACTATTGTTGCTGTAAAACCAATTAATGAATTTATCTGAGAATTAATAT
TATTTACTTTCGCATCTATTTCAGTTTCAGTATAATACCTTTCATCATGATTATGTCCAA
CATTTGATTTTCCACCTAATTTTGAATCAATTTCTGTTTTCTTATAATAATCATTTAAAT
GTTTAACAGCAATATAATTGCCAACATGTACCTGATAACTATTTGCTCCATCTTCACTAG
CTATATTTAAATATAAATCACCAGTAGTTTCATCATGTTCAAATATTGGAGGGTTGGTTT
CATCATTACAATAACACCATAGATTTCCTTCAGCATCTCCTGCTAAAGTGAAAAAACCTG
GTGGAGGTACTGTAATATTTAATAATGGGTTTGCCCATTTGTCGAACAATTCATCAATGG
CTTTACTTACTGTATTGGCATCTGTCTTTAATATGTCGATGTCTAGGAAATCTTCAATGT
TTCCTATTTTTGTGCCTAGGTTTGTTTCTCTTAATCTTGCACATGCATTATTTAATGCTT
TTCTTTCATTTTCAGTTAGTTTAGTCATATTACACTCTCTCGTTTAACATTACCTATTTT
AATCTCTTTGTCACCATTTAGATAAGGATTATAACTTATTTCTGTTATTTGTGCTTTGAA
CACCTCTGAATCTGGGAATCTTACATATAC

It will be appreciated that the optimal concentration of a substance capable of inhibiting the production of methane in a ruminant can be determined using challenge studies in which different concentrations are investigated. In some embodiments, the concentration of the virus capable of inhibiting the production of methane in a ruminant may be >10² pfu/ml, >10³ pfu/ml, >10⁴ pfu/ml, or >10⁶ pfu/ml.

In some embodiments, the composition, bolus, animal lick block, or animal feed provides sustained release of the virus capable of inhibiting the production of methane in vivo in the ruminant. Thus, the composition, bolus or animal lick block delivers predetermined doses of the virus continuously for a predetermined release period, providing a controlled release of the virus to the ruminant. A sustained release composition, bolus, or animal lick block is advantageous because delivery of the virus is provided across the release period, meaning that methane inhibition is sustained for the full release period.

In some embodiments, the dose of the virus is kept constant across the full release period. This is advantageous because it ensures that a constant level of virus is present in the ruminant. Thus, a constant amount of virus will be present in the rumen, resulting in a consistent level of inhibition of methane production.

In other embodiments, the composition, bolus, animal lick block, or animal feed provides intermittent release of the virus. Thus, there are intervals during the release period where no virus is being released to the ruminant. This embodiment allows dosing of the virus capable of inhibiting the production of methane by the ruminant to be spread out and separated with recovery intervals, in order to allow the ruminant time to recover between doses of the virus, if required. In other embodiments the composition of the disclosure comprises two or more viruses capable of inhibiting the production of methane in vivo in a ruminant and is configured to provide release of alternating viruses, each infecting the target Archaea by attaching to the host by binding to different receptors, in order to prevent, or overcome the development of resistance.

In some embodiments, the disclosure relates to an animal lick block comprising the composition of the disclosure, and to methods of reducing the production of methane in a ruminant comprising providing an animal lick block including a virus capable of inhibiting the production of methane in vivo in the ruminant. This is advantageous because animal lick blocks are commonly used to supplement the diet of ruminants with additional nutrients and vitamins. Thus, by providing animal lick blocks which incorporate the compositions of the disclosure, it is possible to administer methane mitigating active agents including a virus to ruminants ad libitum, enabling continuous access to the composition. The composition is then digested by the ruminant and provides controlled release of the active substances in the rumen across the release period.

In some embodiments, the disclosure relates to an animal feed comprising the composition of the disclosure, and to methods of reducing the production of methane in a ruminant comprising providing an animal feed including a virus capable of inhibiting the production of methane in vivo in the ruminant. This is advantageous because animal feeds are used to feed ruminants. Thus, by providing animal feeds which incorporate the compositions of the disclosure, it is possible to administer methane mitigating active agents including a virus to ruminants orally during their normal feeding. The composition is then digested by the ruminant and provides controlled release of the active substances in the rumen across the release period.

The animal feeds and animal lick blocks may be stabilised by formulating them in compositions having a high sugar content, for example greater than 40%, suitably greater than 60%, most suitably greater than 65% and preferably greater than 68%. The high sugar concentration preserves the stability and activity of the virus. Molasses formulations are convenient sugar formulations for use in the compositions of the present disclosure.

In some embodiments, the release period is about one week. In some embodiments, the release period is about one month. In some embodiments, the release period is about one year. In some embodiments, the release period is about 2 years.

Suitably the composition will have a release period selected depending on the expected life of the ruminant. In the case of calves, the release period may thus be between 24 and 36 months, or between 24 to 30 months. In the case of lambs, the release period may be between 6 to 8 months.

In some embodiments, the disclosure relates to a bolus comprising a solid or semi-solid composition, the composition comprising a virus capable of inhibiting the production of methane in vivo in a ruminant, wherein the bolus is configured to provide controlled release of the virus to the ruminant. The bolus may be orally administered to the ruminant. Applicators for administering boluses to ruminants such as cattle are well known to those in the veterinary field. The bolus may be administered to the ruminant after the ruminant reaches an appropriate size. In the case of calves, this may be when they are over 100 kg in weight, and/or about two to three months old. In the case of lambs, this may be when they are over 15 kg in weight and/or about five to eight weeks old.

In some embodiments, one bolus may be administered to deliver a controlled dose over the rest of the calf's life. In other embodiments, a bolus may be administered to provide a dose over an initial period, followed by subsequent boluses. This will depend on the release period for each bolus and the desired total time period over which the virus is delivered to the ruminant.

In some embodiments, the composition, bolus, animal lick block, or animal feed is arranged to provide a predetermined dose of the virus to the ruminant. Thus, the combination of controlled release and predetermined dose of virus allows complete control over the delivery of the virus to the ruminant. This ensures that an effective dose of the virus can be provided to the ruminant across the full release period. The predetermined dose can be adjusted depending on the release characteristics of the composition, bolus or animal lick.

In some embodiments, the disclosure relates to a bolus comprising a solid or semi-solid composition, the composition comprising a virus capable of inhibiting the production of methane in vivo in a ruminant, wherein the bolus is configured to provide controlled release of the virus to the ruminant, and to methods of orally administering these boluses to ruminants.

In some embodiments, the core and the shaft are both dissolvable in liquids found in the rumen. This is advantageous because the bolus will fully dissolve in the rumen of a ruminant.

In some embodiments, the core comprises metal. In some embodiments, the core consists of metal. In some embodiments, the shaft comprises metal. In some embodiments, the shaft consists of metal. In some embodiments, both the core and the shaft comprise metal. In some embodiments, both the core and the shaft consist of metal.

In some embodiments, the majority of the bolus is coated with a coating preventing ingress of liquids such as liquid in the rumen of ruminants, into the bolus. In this arrangement, the ingress of liquid from the ruminant into the bolus can be carefully controlled, because liquid can only enter the bolus through the uncoated portion.

If the bolus comprises a core and a shaft, in some embodiments the bolus is coated on all sides apart from at the top of the shaft, such that fluid can only enter the bolus at the top of the shaft. This results in release of the virus from the bolus at a constant, predetermined rate, as liquids from the rumen ingress into the bolus.

In some embodiments, the bolus further comprises a non-virus containing substance. In some embodiments, the virus capable of inhibiting the production of methane by the ruminant and the non-virus containing substance are arranged such that they are released at different times. In one such arrangement, the virus and the non-virus containing substance are arranged in alternating layers such that, as the bolus dissolves, a dose of virus is released followed by a dose of non-virus containing substance, and so on, to provide intermittent release. In a further embodiment, the bolus comprises two or more viruses capable of inhibiting the production of methane in vivo in a ruminant and is configured to provide release of alternating viruses, each infecting the target Archaea by attaching to the host by binding to different receptors, in order to prevent, or overcome the development of resistance to infection.

In some embodiments of this arrangement, the bolus comprises alternating layers of virus and non-virus containing substance, such that as liquids from the rumen ingress into the bolus, intermittent release is provided. This arrangement is advantageous because the bolus provides controlled release of predetermined doses of a virus capable of inhibiting the production of methane in vivo in the ruminant, thus allowing its administration to be carefully controlled. In some embodiments, the non-virus containing substance comprises one or more feed supplements such as vitamins and minerals and/or therapeutic agents such as anthelmintics.

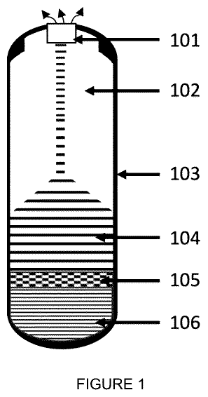

The bolus may comprise a core with a shaft extending therefrom. This arrangement is shown in FIG. 1. The bolus of FIG. 1 comprises an exit port screen 101, a density element 102, and a semipermeable membrane 103. The bolus contains a drug formulation 104 as well as a placebo portion 105, with an osmotic tablet 106.

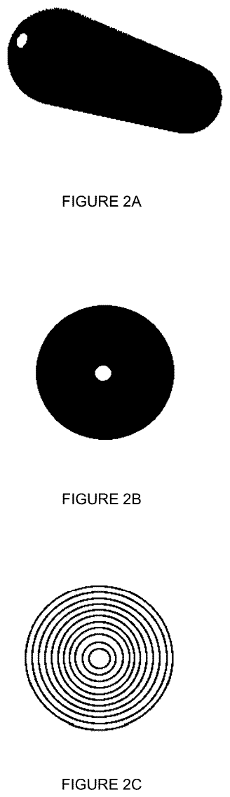

In a further embodiment, the bolus may be tubular, as shown in FIGS. 2a and 2b. This is advantageous for when a shorter duration of release is required, because the surface area available for release of the product is substantially constant without any coating. In this embodiment, the bolus may also incorporate materials to enable the sustained release of the virus over the required release period, such as those described herein. The tubular bolus may have homogenous distribution of virus throughout its structure, or it may have layers of different substances as depicted in FIG. 2c.

In some embodiments, the composition further comprises one or more additional substances capable of inhibiting the production of methane in vivo in a ruminant, selected from brominated aliphatic compounds, carbohydrates, lipids, peptides, further viruses, and phlorotannins. In some embodiments, the composition comprises a combination of at least two viruses. The viruses may be stabilised as discussed above. This is advantageous because archaea and bacteria have evolved numerous defence mechanisms against viruses and are known to develop resistance. It is therefore desirable for preparations to comprise a cocktail of two or more active agents to minimise the impact of such resistance.

In some embodiments, the composition comprises first and second active substances, wherein the first and second active substances are arranged such that they are released at different times. This may be achieved by varying virus composition in different production batches, or by preparing the delivered composition with alternating layers of active substances. In some embodiments, each active substance comprises a combination of two or more viruses. This is advantageous because it further minimises resistance by exposing the ruminant to alternating cycles of different viruses.

### EXAMPLES

Proof of concept was achieved via isolation of viruses with activity against methane-producing archaeal microorganisms and comparison of in vitro methane production by archaeal microorganisms in the presence or absence of these viruses. The methods and experimental results are described as follows.

Methanobacterium bryantii (DSMZ 862) and Methanobrevibacter smithii (DSMZ 861) were purchased from Leibniz Institute DSMZ-German Collection of Microorganisms and cultured as recommended in 119 Broth medium supplemented with rumen fluid in the presence of H²/CO² (0.5-1 bar pressure) anaerobically at 37° C. for 48-72 hrs.

Viruses with activity against Methanobacterium bryantii and Methanobrevibacter smithii were isolated from abattoir specimens of fresh bovine rumen fluid that had been concentrated by filtration and addition of polyethylene glycol (PEG) and sodium chloride to a final concentration of 10% (w/v) and 0.5M, respectively as described below.

First, virus particles were separated from the rumen fluid, concentrated and purified using PEG precipitation and centrifugation. Archaea cultures in 119 Broth were inoculated with the purified virus suspension and the cultures were allowed to grow for 48 hours to allow any Archaea specific viruses to initiate a cycle of infection and amplification.

Following incubation, methane production was measured using gas chromatography-mass spectrometry (GC-MS). Cultures inoculated with the virus preparation were compared to those that were not treated with virus, but were otherwise identical. The presence of active virus was confirmed by a measurable reduction in methane production in cultures exposed to the virus preparation. The viruses released during the infection cycle were recovered from the cultures by filtration with a 0.22 μm filter that allowed the separation of virus particles from the Archaea in the culture medium.

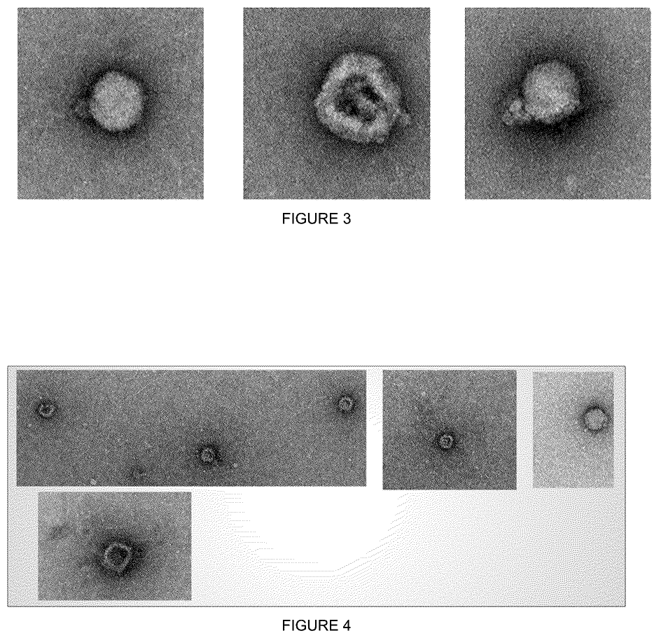

The recovered virus suspension was then used to inoculate a fresh culture of host Archaea cells as before. This process of infection, replication, extraction and re-infection, referred to as passaging, was repeated four and six times to obtain viruses, referred to herein as virus isolates 1 and 2, with activity against Methanobacterium bryantii and Methanobrevibacter smithii respectively. The culture of Methanobacterium bryantii and virus isolate 1 which had been passaged four times to achieve clonal populations of viruses was submitted for transmission electron microscopy. The resulting images are shown in FIG. 3. The culture of Methanobrevibacter smithii and virus isolate 2 were passaged six times to achieve a clonal population and transmission electron microscopy images were obtained. The resulting images are shown in FIG. 4.

The virus with activity against Methanobrevibacter smithii (virus isolate 2) was a novel virus which had the sequence set forth in SEQ ID 1.

### Example 1

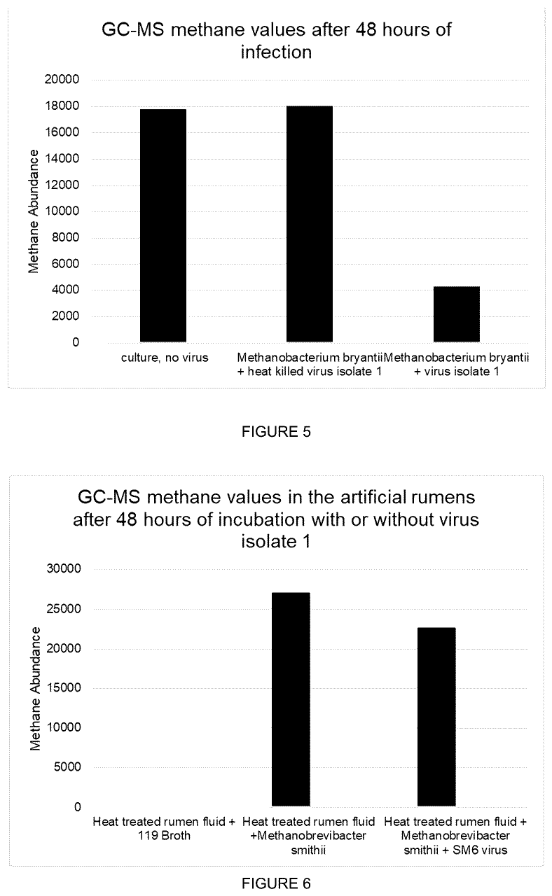

Virus isolate 1 comprising 500 μL of rumen-derived fluid was added to a culture of Methanobacterium bryantii undergoing exponential growth under the recommended growth conditions described above. Heat inactivated virus isolate 1 was added to a second culture and a control culture with an equivalent growth of Methanobacterium bryantii and no added virus was also prepared. The cultures were incubated for 48 hours, then methane production was measured via gas chromatography mass spectrometry (GC-MS). The results of this experiment are shown in Table 1 and FIG. 5.

TABLE 1
GC-MS methane values of gas samples
taken after 48 hours of incubation.

| | GC-MS reading |
|---|---|
| Culture | (m/z = 15) |
| Methanobacterium bryantii culture | 17728 |
| control (no addition of virus) | |
| Methanobacterium bryantii culture with | 17973 |
| the addition of heat inactivated virus isolate 1 | |
| Methanobacterium bryantii culture with | 4268 |
| addition of virus isolate 1 | |

The data from this example demonstrate that a virus isolate with activity against methane producing archaea is capable of reducing their methane production in an ex vivo environment. The lack of inhibition in the heat inactivated sample further demonstrates that an active virus is necessary for the inhibition of methane production.

### Example 2

The rumen serves as a chamber, providing a hydrogen-rich anaerobic environment for the microbial fermentation of ingesta. This environment was replicated with an apparatus comprising a Hungate tube, to provide an airtight environment, which contained bovine rumen fluid. The rumen fluid for these experiments was obtained from animals within 30 minutes of euthanasia for prompt use. The chamber was supplied with a constant H₂/CO₂ gas mixture at a ratio of 80/20% (v/v) respectively to establish identical anaerobic conditions to those of the bovine rumen. Individual flasks received a fixed volume of either:
(i) heat-treated rumen fluid with 119 Broth medium
(ii) heat-treated rumen fluid plus a live culture of Methanobrevibacter smithii in 119 Broth medium
(iii) heat-treated rumen fluid plus a live culture of Methanobrevibacter smithii in 119 Broth medium as well as virus isolate 1.

Heat treatment of the rumen fluid was carried out at 80° C. for 5 minutes. In each instance, the majority (60%) of the fluid within the apparatus comprised heat-treated rumen fluid. Each treatment underwent anaerobic incubation in the presence of a H₂/CO₂ gas mix (0.5-1 bar pressure) at 37° C. for 48 hours. Subsequently, the gas samples from each artificial rumen were analysed by GC-MS, to enable the comparison of methane concentrations in each artificial rumen. The results of this experiment are shown in Table 2 and FIG. 6.

TABLE 2
GC-MS methane values after 48 h of incubation

| | GC-MS reading |
|---|---|
| Culture | (m/z = 15) |
| Heat treated rumen fluid | Not detected |
| Heat treated rumen fluid + Methanobrevibacter | 27020 |
| smithii | |
| Heat treated rumen fluid + | 22665 |
| Methanobrevibacter smithii + virus isolate 2 | |

The results show that in the experiments employing the rumen model in vitro, the heat-treated rumen fluid produced no methane. In contrast, rumen fluid with a live population of archaea microorganisms resulted in methane emission. However, this emission was measurably reduced by the presence of virus isolate 2, which specifically infects Methanobrevibacter smithii that was present in the rumen fluid. Therefore, it is possible to mitigate methane emission in the rumen of cattle and other ruminants by introducing selected anti-archaeal viruses.

### NUMBERED EMBODIMENTS OF THE DISCLOSURE

1. A solid or semi-solid composition for administration to a ruminant comprising a substance capable of inhibiting the production of methane in vivo in the ruminant.
2. A bolus comprising a solid or semi-solid composition, the composition comprising a substance capable of inhibiting the production of methane in vivo in a ruminant, wherein the bolus is configured to provide controlled release of the substance to the ruminant.
3. A solid or semi-solid animal lick block comprising a composition comprising a substance capable of inhibiting the production of methane in vivo in a ruminant.
4. An animal feed comprising a composition comprising a substance capable of inhibiting the production of methane in vivo in a ruminant.
5. A method of reducing the production of methane in a ruminant comprising orally administering a bolus to the ruminant, wherein the bolus comprises a substance capable of inhibiting the production of methane in vivo in the ruminant and provides controlled release of the substance to the ruminant.
6. A method of reducing the production of methane in a ruminant, comprising providing an animal lick block or animal feed which includes a substance capable of inhibiting the production of methane in vivo in the ruminant, wherein the substance is orally transferred to the ruminant when the ruminant consumes and digests the components of the lick block or animal feed.
7. A composition according to embodiment 3 or a method according to embodiment 6, wherein the substance is covalently linked to one or more components of the animal lick block.
8. A composition, bolus, animal lick block, animal feed or method according to any preceding embodiment, wherein the ruminant is a bovine, ovine, caprine, or elaphine animal, preferably a bovine animal.
9. A composition, bolus, animal lick block, animal feed or method according to any preceding embodiment, wherein the composition is a solid composition.
10. A composition, bolus, animal lick block, animal feed or method according to any preceding embodiment, wherein the composition is a semi-solid composition, preferably a gel or colloid.
11. A bolus or method according to any preceding embodiment, wherein the controlled release is sustained release.
12. A bolus or method according to any preceding embodiment, wherein the controlled release is intermittent release.
13. A composition, bolus, animal lick block, animal feed or method according to any preceding embodiment, wherein the composition is adapted to release a predetermined dose of the substance to the ruminant.
14. A composition, bolus, animal lick block, animal feed or method according to any preceding embodiment, wherein the substance comprises an inhibitor involved in the metabolic pathway of methane production in the rumen.
15. A composition, bolus, animal lick block, animal feed or method according to any preceding embodiment, wherein the substance comprises an inhibitor of methanogens.
16. A composition, bolus, animal lick block, animal feed or method according to any preceding embodiment, wherein the substance is capable of disrupting the enzymes of gut microbes in the rumen.
17. A composition, bolus, animal lick block, animal feed or method according to any preceding embodiment, wherein the substance comprises a brominated aliphatic compound, a carbohydrate, a lipid, a peptide, a virus, a phlorotannin or a combination thereof.
18. A composition, bolus, animal lick block, animal feed or method according to any preceding embodiment, wherein the substance comprises bromoform, 3-nitroxypropanol, hydoxypropionic acid, a virus or a combination thereof.
19. A composition, bolus, animal lick block, animal feed or method according to any preceding embodiment, wherein the substance comprises bromoform.
20. A composition, bolus, animal lick block, animal feed or method according to any preceding embodiment, wherein the substance comprises a virus.
21. A composition, bolus, animal lick block, animal feed or method according to embodiment 19, wherein the virus is a virus which targets metabolic processes involved in methanogenesis.
22. A composition, bolus, animal lick block, animal feed or method according to embodiment 20, wherein the virus is stabilised.
23. A composition, bolus, animal lick block, animal feed or method according to embodiment 22, wherein the virus is stabilised by immobilisation to a substrate.
24. A composition, bolus, animal lick block, animal feed or method according to embodiment 23, wherein the virus is immobilised via its head.
25. A composition, bolus, animal lick block, animal feed or method according to any preceding embodiment, wherein the substance comprises a combination of at least two brominated aliphatic compounds, carbohydrates, lipids, peptides, viruses and phlorotannins.
26. A composition, bolus, animal lick block, animal feed or method according to embodiment 24, wherein the substance comprises a combination of at least two viruses, preferably wherein the at least two viruses are stabilised.
27. A composition, bolus, animal lick block, animal feed or method according to any preceding embodiment, wherein the composition further comprises an inactive substance.
28. A composition, bolus, animal lick block, animal feed or method according to any preceding embodiment, wherein the composition further comprises a second substance capable of inhibiting the production of methane in vivo in the ruminant.
29. A composition, bolus, animal lick block, animal feed or method according to embodiment 27 or 28, wherein the substance capable of inhibiting the production of methane by the ruminant and the inactive substance and/or second substance capable of inhibiting the production of methane in vivo in the ruminant are arranged such that they are released at different times.
30. A bolus according to any preceding embodiment, wherein the majority of the bolus is coated with a coating that prevents the ingress of liquids into the bolus.
31. A bolus according to embodiment 30, wherein the bolus comprises a core with a shaft extending therefrom and the substance capable of inhibiting the production of methane by the ruminant is arranged around the shaft.
32. A bolus according to embodiment 31, wherein the bolus further comprises an inactive substance and wherein the substance capable of inhibiting the production of methane by the ruminant and the inactive substance are arranged in layers around the shaft of the bolus.
33. A bolus according to embodiment 31 or 32, wherein the core and shaft of the bolus are dissolvable in liquids found in the rumen of ruminants.

The foregoing disclosure provides exemplary embodiments of the disclosure and is not intended to be limiting. It will be appreciated that various other modifications and variations of the disclosure are also possible.

1. A solid or semi-solid composition comprising a virus capable of reducing methane production in a ruminant.

2. An animal lick block comprising the solid or semi-solid composition according to claim 1.

3. An animal feed comprising the solid or semi-solid composition according to claim 1.

4. A bolus comprising the solid or semi-solid composition according to claim 1, wherein the bolus is configured to provide controlled release of the virus to the ruminant.

5. The solid or semi-solid composition according to claim 2 wherein the virus is covalently linked to one or more components of the animal lick block.

6. The solid or semi-solid composition according to claim 1, wherein the virus targets archaea involved in methane production in ruminants.

7. The solid or semi-solid composition according to claim 1, wherein the composition is a solid, a gel, or a colloid.

8. The solid or semi-solid composition according to claim 1, wherein the composition is configured to provide controlled release of the virus to the ruminant.

9. The solid or semi-solid composition according to claim 1, wherein the composition is capable of releasing a predetermined dose of the virus to the ruminant.

10. The solid or semi-solid composition according to claim 1, wherein the virus is immobilized to a substrate.

11. The solid or semi-solid composition according to claim 1, wherein the composition further comprises a combination of the virus and one or more of: a brominated aliphatic compound, a carbohydrate, a lipid, a peptide, a phlorotannin, and one or more additional viruses.

12. The solid or semi-solid composition according to claim 1, wherein the composition further comprises a non-viral substance and/or a second substance capable of reducing methane production in the ruminant.

13. The solid or semi-solid composition according to claim 12, wherein the composition is configured to release the virus and the non-viral substance and/or second substance at different times.

14. The solid or semi-solid bolus composition according to claim 4, wherein at least 50% of the bolus is coated with a coating that prevents the ingress of liquids into the bolus.

15. The solid or semi-solid bolus composition according to claim 14, wherein the bolus comprises a core with a shaft extending therefrom and the substance capable of reducing methane production is arranged around the shaft.

16. The solid or semi-solid bolus composition according to claim 15, wherein the bolus further comprises a non-viral substance and wherein the virus and the non-viral substance are arranged in layers around the shaft of the bolus.

17. A method of reducing methane production in a ruminant, comprising orally administering to the ruminant a solid or semi-solid composition comprising an effective amount of a virus capable of reducing methane production in the ruminant.

18. The method according to claim 17, wherein the solid or semi-solid composition is a bolus that provides controlled release of the virus to the ruminant.

19. The method according to claim 17, wherein the solid or semi-solid composition is an animal lick block or an animal feed, and the virus is administered to the ruminant by its consumption or digestion of the lick block or animal feed.

20. A virus capable of inhibiting the production of methane in vivo in the ruminant, wherein the virus targets archaea which are involved in the production of methane in ruminants.
